SEQUENCE LISTING (45 sequences)

SEQ ID NO: 1 - RNA, 19 nt, Artificial Sequence
  Single-stranded oligonucleotide
  misc_feature (1)..(1): phospho, 2′-methyl
  misc_feature (2)..(2): 2′fluoro
  modified_base (3)..(3): gm
  misc_feature (4)..(4): 2′fluoro
  misc_feature (5)..(5): 2′methyl
  misc_feature (6)..(6): 2′fluoro
  modified_base (7)..(7): cm
  misc_feature (8)..(8): 2′fluoro
  modified_base (9)..(9): um
  misc_feature (10)..(10): 2′fluoro
  modified_base (11)..(11): gm
  misc_feature (12)..(12): 2′fluoro
  misc_feature (13)..(13): 2′methyl
  misc_feature (14)..(14): 2′fluoro
  misc_feature (15)..(15): 2′methyl
  misc_feature (16)..(16): 2′fluoro
  misc_feature (17)..(17): 2′methyl
  misc_feature (18)..(18): 2′fluoro
  modified_base (19)..(19): cm
uuguacccuaggaaauacc

SEQ ID NO: 2 - RNA, 19 nt, Artificial Sequence
  Single-stranded oligonucleotide
  misc_feature (1)..(1): 2′ fluoro
  modified_base (2)..(2): gm
  misc_feature (3)..(3): 2′ fluoro
  misc_feature (4)..(4): 2′methyl
  misc_feature (5)..(5): 2′ fluoro
  modified_base (6)..(6): um
  misc_feature (7)..(7): 2′ fluoro
  misc_feature (8)..(8): 2′ fluoro
  misc_feature (9)..(9): 2′ fluoro
  modified_base (10)..(10): um
  misc_feature (11)..(11): 2′ fluoro
  modified_base (12)..(12): gm
  misc_feature (13)..(13): 2′ fluoro
  modified_base (14)..(14): gm
  misc_feature (15)..(15): 2′ fluoro
  misc_feature (16)..(16): 2′methyl
  misc_feature (17)..(17): 2′ fluoro
  misc_feature (18)..(18): 2′methyl
  misc_feature (19)..(19): 2′ fluoro, NHC6 linker, trivalent N- acetylgalactosamine
gguauuuccuaggguacaa

SEQ ID NO: 3 - RNA, 19 nt, Artificial Sequence
  Single-stranded oligonucleotide
  misc_feature (1)..(1): phosphate, 2′methyl
  misc_feature (2)..(2): 2′fluoro
  modified_base (3)..(3): cm
  misc_feature (4)..(4): 2′fluoro
  misc_feature (5)..(5): 2′methyl
  misc_feature (6)..(6): 2′fluoro
  misc_feature (7)..(7): 2′methyl
  misc_feature (8)..(8): 2′fluoro
  modified_base (9)..(9): gm
  misc_feature (10)..(10): 2′fluoro
  misc_feature (11)..(11): 2′methyl
  misc_feature (12)..(12): 2′fluoro
  modified_base (13)..(13): cm
  misc_feature (14)..(14): 2′fluoro
  modified_base (15)..(15): gm
  misc_feature (16)..(16): 2′fluoro
  modified_base (17)..(17): um
  misc_feature (18)..(18): 2′fluoro
  misc_feature (19)..(19): 2′methyl
aaccagaagaagcagguga

SEQ ID NO: 4 - RNA, 19 nt, Artificial Sequence
  Single-stranded oligonucleotide
  misc_feature (1)..(1): 2′fluoro
  modified_base (2)..(2): cm
  misc_feature (3)..(3): 2′fluoro
  modified_base (4)..(4): cm
  misc_feature (5)..(5): 2′fluoro
  modified_base (6)..(6): um
  misc_feature (7)..(7): 2′fluoro
  misc_feature (8)..(8): 2′fluoro
  misc_feature (9)..(9): 2′fluoro
  modified_base (10)..(10): um
  misc_feature (11)..(11): 2′fluoro
  modified_base (12)..(12): um
  misc_feature (13)..(13): 2′fluoro
  modified_base (14)..(14): cm
  misc_feature (15)..(15): 2′fluoro
  modified_base (16)..(16): gm
  misc_feature (17)..(17): 2′fluoro
  modified_base (18)..(18): um
  misc_feature (19)..(19): 2′ fluoro, NHC6-linker, trivalent N- acetylgalactosamine
ucaccugcuucuucugguu

SEQ ID NO: 5 - RNA, 19 nt, Artificial Sequence
  Single-stranded oligonucleotide
  misc_feature (1)..(1): phospho, 2′methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (16)..(16): 2′ fluoro
  modified_base (17)..(17): gm
  misc_feature (18)..(18): 2′ fluoro
  modified_base (19)..(19): cm
gcaucuucugggcuuuggc

SEQ ID NO: 6 - RNA, 19 nt, Artificial Sequence
  Single-stranded oligonucleotide
  misc_feature (1)..(1): 2′ fluoro
  modified_base (2)..(2): cm
  misc_feature (3)..(3): 2′ fluoro
  misc_feature (4)..(4): 2′ methyl
  misc_feature (5)..(5): 2′ fluoro
  misc_feature (6)..(6): 2′ methyl
  misc_feature (7)..(7): 2′ fluoro
  misc_feature (8)..(8): 2′ fluoro
  misc_feature (9)..(9): 2′ fluoro
  modified_base (10)..(10): cm
  misc_feature (11)..(11): 2′ fluoro
  modified_base (12)..(12): gm
  misc_feature (13)..(13): 2′ fluoro
  misc_feature (14)..(14): 2′ methyl
  misc_feature (15)..(15): 2′ fluoro
  misc_feature (16)..(16): 2′ methyl
  misc_feature (17)..(17): 2′ fluoro
  modified_base (18)..(18): gm
  misc_feature (19)..(19): 2′ fluoro, NHC6-linker, trivalent N- acetylgalactosamine
gccaaagcccagaagaugc

SEQ ID NO: 7 - RNA, 19 nt, Artificial Sequence
  Single-stranded oligonucleotide
  misc_feature (1)..(1): phosphate, 2′methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (16)..(16): 2′ fluoro
  modified_base (17)..(17): cm
  misc_feature (18)..(18): 2′ fluoro
  misc_feature (19)..(19): 2′ methyl
uguacccuaggaaauacca

SEQ ID NO: 8 - RNA, 19 nt, Artificial Sequence
  Single-stranded oligonucleotide
  misc_feature (1)..(1): 2′ fluoro
  modified_base (2)..(2): gm
  misc_feature (3)..(3): 2′ fluoro
  modified_base (4)..(4): um
  misc_feature (5)..(5): 2′ fluoro
  modified_base (6)..(6): um
  misc_feature (7)..(7): 2′ fluoro
  misc_feature (8)..(8): 2′ fluoro
  misc_feature (9)..(9): 2′ fluoro
  modified_base (10)..(10): cm
  misc_feature (11)..(11): 2′ fluoro
  misc_feature (12)..(12): 2′ methyl
  misc_feature (13)..(13): 2′ fluoro
  modified_base (14)..(14): gm
  misc_feature (15)..(15): 2′ fluoro
  modified_base (16)..(16): um
  misc_feature (17)..(17): 2′ fluoro
  modified_base (18)..(18): cm
  misc_feature (19)..(19): 2′ fluoro, NHC6-linker, trivalent N- acetylgalactosamine
ugguauuuccuaggguaca

SEQ ID NO: 9 - RNA, 19 nt, Artificial Sequence
  Single-stranded oligonucleotide
  misc_feature (1)..(1): phosphate, 2′ methyl
  misc_feature (2)..(2): 2′ fluoro
  modified_base (3)..(3): cm
  misc_feature (4)..(4): 2′ fluoro
  modified_base (5)..(5): gm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): um
  misc_feature (12)..(12): 2′ fluoro
  modified_base (13)..(13): gm
  misc_feature (14)..(14): 2′ fluoro
  modified_base (15)..(15): cm
  misc_feature (16)..(16): 2′ fluoro
  modified_base (17)..(17): cm
  misc_feature (18)..(18): 2′ fluoro
  modified_base (19)..(19): gm
cacagaugugucgaccccg

SEQ ID NO: 10 - RNA, 19 nt, Artificial Sequence
  Single-stranded oligonucleotide
  misc_feature (1)..(1): 2′ fluoro
  modified_base (2)..(2): gm
  misc_feature (3)..(3): 2′ fluoro
  modified_base (4)..(4): gm
  misc_feature (5)..(5): 2′ fluoro
  modified_base (6)..(6): um
  misc_feature (7)..(7): 2′ fluoro
  misc_feature (8)..(8): 2′ fluoro
  misc_feature (9)..(9): 2′ fluoro
  modified_base (10)..(10): cm
  misc_feature (11)..(11): 2′ fluoro
  modified_base (12)..(12): cm
  misc_feature (13)..(13): 2′ fluoro
  modified_base (14)..(14): um
  misc_feature (15)..(15): 2′ fluoro
  modified_base (16)..(16): um
  misc_feature (17)..(17): 2′ fluoro
  modified_base (18)..(18): um
  misc_feature (19)..(19): 2′ fluoro, NHC6-linker, trivalent N- acetylgalactosamine
cggggucgacacaucugug

SEQ ID NO: 11 - RNA, 34 nt, Artificial Sequence
  Single-stranded oligonucleotide
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  modified_base (15)..(15): gm
  misc_feature (15)..(19): phosphorothioate linker
  misc_feature (16)..(16): 2′ fluoro
  modified_base (17)..(17): um
  misc_feature (18)..(18): 2′ fluoro
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine
aaccagaagaagcaggugaaagcccagaagaugc

SEQ ID NO: 12 - RNA, 34 nt, Artificial Sequence
  Single-stranded oligonucleotide
  misc_feature (1)..(1): phosphate, 2′ methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): gm
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine
gcaucuucugggcuuuggccugcuucuucugguu

SEQ ID NO: 13 - RNA, 34 nt, Artificial Sequence; identical to SEQ ID NO: 11
  Single-stranded oligonucleotide
  misc_feature (1)..(1): phosphate

SEQ ID NO: 14 - RNA, 34 nt, Artificial Sequence; identical to SEQ ID NO: 12
  Single-stranded oligonucleotide
  misc_feature (1)..(1): phosphate

SEQ ID NO: 15 - RNA, 34 nt, Artificial Sequence
  Single-stranded oligonucleotide
  misc_feature (1)..(1): phosphate, 2′ methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): gm
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  misc_feature (27)..(27): 2′ methyl
  misc_feature (28)..(28): 2′ fluoro
  modified_base (29)..(29): gm
  misc_feature (30)..(30): 2′ fluoro
  modified_base (31)..(31): um
  misc_feature (32)..(32): 2′ fluoro
  modified_base (33)..(33): cm
  misc_feature (34)..(34): 2′ fluoro, NHC6-linker, trivalent N- acetylgalactosamine
gcaucuucugggcuuuggcauuuccuaggguaca

SEQ ID NO: 16 - RNA, 34 nt, Artificial Sequence
  Single-stranded oligonucleotide
  misc_feature (1)..(1): phosphate, 2′ methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): cm
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine
uguacccuaggaaauaccacugcuucuucugguu

SEQ ID NO: 17 - RNA, 34 nt, Artificial Sequence; identical to SEQ ID NO: 15
  Single-stranded oligonucleotide
  misc_feature (1)..(1): phosphate

SEQ ID NO: 18 - RNA, 34 nt, Artificial Sequence; identical to SEQ ID NO: 16
  Single-stranded oligonucleotide
  misc_feature (1)..(1): phosphate

SEQ ID NO: 19 - RNA, 34 nt, Artificial Sequence
  Single-stranded oligonucleotide
  misc_feature (1)..(1): phosphate, 2′ methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): cm
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): cm
  misc_feature (28)..(28): 2′ fluoro
  modified_base (29)..(29): um
  misc_feature (30)..(30): 2′ fluoro
  modified_base (31)..(31): um
  misc_feature (32)..(32): 2′ fluoro
  modified_base (33)..(33): um
  misc_feature (34)..(34): 2′ fluoro, NHC6-linker, trivalent N- acetylgalactosamine
uguacccuaggaaauaccagucgacacaucugug

SEQ ID NO: 20 - RNA, 34 nt, Artificial Sequence
  Single-stranded oligonucleotide
  misc_feature (1)..(1): phosphate, 2′ methyl
  misc_feature (2)..(2): 2′ fluoro
  modified_base (3)..(3): cm
  misc_feature (4)..(4): 2′ fluoro
  modified_base (5)..(5): gm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): um
  misc_feature (12)..(12): 2′ fluoro
  modified_base (13)..(13): gm
  misc_feature (14)..(14): 2′ fluoro
  misc_feature (15)..(19): phosphorothioate linkage
  modified_base (15)..(15): cm
  misc_feature (16)..(16): 2′ fluoro
  modified_base (17)..(17): cm
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): gm
  misc_feature (28)..(28): 2′ fluoro
  modified_base (29)..(29): gm
  misc_feature (30)..(30): 2′ fluoro
  misc_feature (31)..(31): 2′ methyl
  misc_feature (32)..(32): 2′ fluoro
  misc_feature (33)..(33): 2′ methyl
  misc_feature (34)..(34): 2′ fluoro, NHC6-linker, trivalent N- acetylgalactosamine
cacagaugugucgaccccguuuccuaggguacaa

SEQ ID NO: 21 - RNA, 34 nt, Artificial Sequence
  Single-stranded oligonucleotide
  misc_feature (1)..(1): phosphate, 2′methyl
  misc_feature (2)..(2): 2′ fluoro
  modified_base (3)..(3): gm
  misc_feature (4)..(4): 2′ fluoro
  misc_feature (5)..(5): 2′ methyl
  misc_feature (6)..(6): 2′ fluoro
  modified_base (7)..(7): cm
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  misc_feature (15)..(15): 2′ methyl
  misc_feature (15)..(19): phosphorothioate linkage
  misc_feature (16)..(16): 2′ fluoro
  misc_feature (17)..(17): 2′ methyl
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine
uuguacccuaggaaauacccugcuucuucugguu

SEQ ID NO: 22 - RNA, 34 nt, Artificial Sequence; identical to SEQ ID NO: 19
  Single-stranded oligonucleotide
  misc_feature (1)..(1): phosphate

SEQ ID NO: 23 - RNA, 34 nt, Artificial Sequence; identical to SEQ ID NO: 20
  Single-stranded oligonucleotide
  misc_feature (1)..(1): phosphate

SEQ ID NO: 24 - RNA, 34 nt, Artificial Sequence; identical to SEQ ID NO: 21
  Single-stranded oligonucleotide
  misc_feature (1)..(1): phosphate

SEQ ID NO: 25 - RNA, 34 nt, Artificial Sequence; identical to SEQ ID NO: 11
  Single-stranded oligonucleotide
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  modified_base (15)..(15): gm
  misc_feature (15)..(19): phosphorothioate linkage
  misc_feature (16)..(16): 2′ fluoro
  modified_base (17)..(17): um
  misc_feature (18)..(18): 2′ fluoro
  misc_feature (19)..(19): 2′ methyl
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine

SEQ ID NO: 26 - RNA, 34 nt, Artificial Sequence; identical to SEQ ID NO: 12
  Single-stranded oligonucleotide
  misc_feature (1)..(1): phosphate, 2′ methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): gm
  misc_feature (18)..(18): 2′ fluoro
  modified_base (19)..(19): cm
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine

SEQ ID NO: 27 - RNA, 34 nt, Artificial Sequence; identical to SEQ ID NO: 15
  Single-stranded oligonucleotide
  misc_feature (1)..(1): phosphate, 2′ methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): gm
  misc_feature (18)..(18): 2′ fluoro
  modified_base (19)..(19): cm
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  misc_feature (27)..(27): 2′ methyl
  misc_feature (28)..(28): 2′ fluoro
  modified_base (29)..(29): gm
  misc_feature (30)..(30): 2′ fluoro
  modified_base (31)..(31): um
  misc_feature (32)..(32): 2′ fluoro
  modified_base (33)..(33): cm
  misc_feature (34)..(34): 2′ fluoro, NHC6-linker, trivalent N- acetylgalactosamine

SEQ ID NO: 28 - RNA, 34 nt, Artificial Sequence; identical to SEQ ID NO: 16
  Single-stranded oligonucleotide
  misc_feature (1)..(1): phosphate, 2′methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): cm
  misc_feature (18)..(18): 2′ fluoro
  misc_feature (19)..(19): 2′ methyl
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine

SEQ ID NO: 29 - RNA, 34 nt, Artificial Sequence; identical to SEQ ID NO: 19
  Single-stranded oligonucleotide
  misc_feature (1)..(1): phosphate, 2′ methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (15)..(19): phosphorothioate linkage
  misc_feature (16)..(16): 2′ fluoro
  modified_base (17)..(17): cm
  misc_feature (18)..(18): 2′ fluoro
  misc_feature (19)..(19): 2′ methyl
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): cm
  misc_feature (28)..(28): 2′ fluoro
  modified_base (29)..(29): um
  misc_feature (30)..(30): 2′ fluoro
  modified_base (31)..(31): um
  misc_feature (32)..(32): 2′ fluoro
  modified_base (33)..(33): um
  misc_feature (34)..(34): 2′ fluoro, NHC6-linker, trivalent N- acetylgalactosamine

SEQ ID NO: 30 - RNA, 34 nt, Artificial Sequence; identical to SEQ ID NO: 20
  Single-stranded oligonucleotide
  misc_feature (1)..(1): phosphate, 2′ methyl
  misc_feature (2)..(2): 2′ fluoro
  modified_base (3)..(3): cm
  misc_feature (4)..(4): 2′ fluoro
  modified_base (5)..(5): gm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): um
  misc_feature (12)..(12): 2′ fluoro
  modified_base (13)..(13): gm
  misc_feature (14)..(14): 2′ fluoro
  modified_base (15)..(15): cm
  misc_feature (15)..(19): phosphorothioate linkage
  misc_feature (16)..(16): 2′ fluoro
  modified_base (17)..(17): cm
  misc_feature (18)..(18): 2′ fluoro
  modified_base (19)..(19): gm
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): gm
  misc_feature (28)..(28): 2′ fluoro
  modified_base (29)..(29): gm
  misc_feature (30)..(30): 2′ fluoro
  misc_feature (31)..(31): 2′ methyl
  misc_feature (32)..(32): 2′ fluoro
  misc_feature (33)..(33): 2′ methyl
  misc_feature (34)..(34): 2′ fluoro, NHC6-linker, trivalent N- acetylgalactosamine

SEQ ID NO: 31 - RNA, 34 nt, Artificial Sequence; identical to SEQ ID NO: 21
  Single-stranded oligonucleotide
  misc_feature (1)..(1): phosphate, 2′ methyl
  misc_feature (2)..(2): 2′ fluoro
  modified_base (3)..(3): gm
  misc_feature (4)..(4): 2′ fluoro
  misc_feature (5)..(5): 2′ methyl
  misc_feature (6)..(6): 2′ fluoro
  modified_base (7)..(7): cm
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  misc_feature (15)..(15): 2′ methyl
  misc_feature (15)..(19): phosphorothioate linkage
  misc_feature (16)..(16): 2′ fluoro
  misc_feature (17)..(17): 2′ methyl
  misc_feature (18)..(18): 2′ fluoro
  modified_base (19)..(19): cm
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine

SEQ ID NO: 32 - RNA, 38 nt, Artificial Sequence
  construct - conventional #8 (XD-16853)
  misc_feature (1)..(1): phosphate, 2′methyl
  misc_feature (2)..(2): 2′ fluoro
  modified_base (3)..(3): gm
  misc_feature (4)..(4): 2′ fluoro
  misc_feature (5)..(5): 2′ methyl
  misc_feature (6)..(6): 2′ fluoro
  modified_base (7)..(7): cm
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  misc_feature (15)..(15): 2′ methyl
  misc_feature (16)..(16): 2′ fluoro
  misc_feature (17)..(17): 2′ methyl
  misc_feature (18)..(18): 2′ fluoro
  modified_base (19)..(19): cm
  misc_feature (20)..(20): 2′ fluoro
  modified_base (21)..(21): gm
  misc_feature (22)..(22): 2′ fluoro
  misc_feature (23)..(23): 2′ methyl
  misc_feature (24)..(24): 2′ fluoro
  modified_base (25)..(25): um
  misc_feature (26)..(26): 2′ fluoro
  misc_feature (27)..(27): 2′ fluoro
  misc_feature (28)..(28): 2′ fluoro
  modified_base (29)..(29): um
  misc_feature (30)..(30): 2′ fluoro
  modified_base (31)..(31): gm
  misc_feature (32)..(32): 2′ fluoro
  modified_base (33)..(33): gm
  misc_feature (34)..(34): 2′ fluoro
  misc_feature (35)..(35): 2′ methyl
  misc_feature (36)..(36): 2′ fluoro
  misc_feature (37)..(37): 2′ methyl
  misc_feature (38)..(38): 2′ fluoro, NHC6-linker, trivalent N- acetylgalactosamine
uuguacccuaggaaauaccgguauuuccuaggguacaa

SEQ ID NO: 33 - RNA, 38 nt, Artificial Sequence
  construct conventional #9 (XD-16854)
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  modified_base (15)..(15): gm
  misc_feature (16)..(16): 2′ fluoro
  modified_base (17)..(17): um
  misc_feature (18)..(18): 2′ fluoro
  misc_feature (19)..(19): 2′ methyl
  misc_feature (20)..(20): 2′ fluoro
  modified_base (21)..(21): cm
  misc_feature (22)..(22): 2′ fluoro
  modified_base (23)..(23): cm
  misc_feature (24)..(24): 2′ fluoro
  modified_base (25)..(25): um
  misc_feature (26)..(26): 2′ fluoro
  misc_feature (27)..(27): 2′ fluoro
  misc_feature (28)..(28): 2′ fluoro
  modified_base (29)..(29): um
  misc_feature (30)..(30): 2′ fluoro
  modified_base (31)..(31): um
  misc_feature (32)..(32): 2′ fluoro
  modified_base (33)..(33): cm
  misc_feature (34)..(34): 2′ fluoro
  modified_base (35)..(35): gm
  misc_feature (36)..(36): 2′ fluoro
  modified_base (37)..(37): um
  misc_feature (38)..(38): 2′ fluoro, NHC6-linker, trivalent N- acetylgalactosamine
aaccagaagaagcaggugaucaccugcuucuucugguu

SEQ ID NO: 34 - RNA, 38 nt, Artificial Sequence
  construct conventional #12 (XD-16855)
  misc_feature (1)..(1): phosphate, 2′ methyl
  misc_feature (2)..(2): 2′ fluoro
  misc_feature (3)..(3): 2′ methyl
  misc_feature (4)..(4): 2′ fluoro
  modified_base (5)..(5): cm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  modified_base (13)..(13): cm
  misc_feature (14)..(14): 2′ fluoro
  modified_base (15)..(15): um
  misc_feature (16)..(16): 2′ fluoro
  modified_base (17)..(17): gm
  misc_feature (18)..(18): 2′ fluoro
  modified_base (19)..(19): cm
  misc_feature (20)..(20): 2′ fluoro
  modified_base (21)..(21): cm
  misc_feature (22)..(22): 2′ fluoro
  misc_feature (23)..(23): 2′ methyl
  misc_feature (24)..(24): 2′ fluoro
  misc_feature (25)..(25): 2′ methyl
  misc_feature (26)..(26): 2′ fluoro
  misc_feature (27)..(27): 2′ fluoro
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  misc_feature (33)..(33): 2′ methyl
  misc_feature (34)..(34): 2′ fluoro
  misc_feature (35)..(35): 2′ methyl
  misc_feature (36)..(36): 2′ fluoro
  modified_base (37)..(37): gm
  misc_feature (38)..(38): 2′ fluoro, NHC6-linker, trivalent N- acetylgalactosamine
gcaucuucugggcuuuggcgccaaagcccagaagaugc

SEQ ID NO: 35 - RNA, 38 nt, Artificial Sequence
  construct conventioal #15 (XD-16856)
  misc_feature (1)..(1): phosphate, 2′methyl
  misc_feature (2)..(2): 2′ fluoro
  modified_base (3)..(3): um
  misc_feature (4)..(4): 2′ fluoro
  modified_base (5)..(5): cm
  misc_feature (6)..(6): 2′ fluoro
  modified_base (7)..(7): cm
  misc_feature (8)..(8): 2′ fluoro
  misc_feature (9)..(9): 2′ methyl
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  modified_base (15)..(15): um
  misc_feature (16)..(16): 2′ fluoro
  modified_base (17)..(17): cm
  misc_feature (18)..(18): 2′ fluoro
  misc_feature (19)..(19): 2′ methyl
  misc_feature (20)..(20): 2′ fluoro
  modified_base (21)..(21): gm
  misc_feature (22)..(22): 2′ fluoro
  modified_base (23)..(23): um
  misc_feature (24)..(24): 2′ fluoro
  modified_base (25)..(25): um
  misc_feature (26)..(26): 2′ fluoro
  misc_feature (27)..(27): 2′ fluoro
  misc_feature (28)..(28): 2′ fluoro
  modified_base (29)..(29): cm
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro
  modified_base (35)..(35): um
  misc_feature (36)..(36): 2′ fluoro
  modified_base (37)..(37): cm
  misc_feature (38)..(38): 2′ fluoro, NHC6-linker, trivalent N- acetylgalactosamine
uguacccuaggaaauaccaugguauuuccuaggguaca

SEQ ID NO: 36 - RNA, 38 nt, Artificial Sequence
  construct conventional #14 (XD-16857)
  misc_feature (1)..(1): phosphate, 2′methyl
  misc_feature (2)..(2): 2′ fluoro
  modified_base (3)..(3): cm
  misc_feature (4)..(4): 2′ fluoro
  modified_base (5)..(5): gm
  misc_feature (6)..(6): 2′ fluoro
  modified_base (7)..(7): um
  misc_feature (8)..(8): 2′ fluoro
  modified_base (9)..(9): um
  misc_feature (10)..(10): 2′ fluoro
  modified_base (11)..(11): um
  misc_feature (12)..(12): 2′ fluoro
  modified_base (13)..(13): gm
  misc_feature (14)..(14): 2′ fluoro
  modified_base (15)..(15): cm
  misc_feature (16)..(16): 2′ fluoro
  modified_base (17)..(17): cm
  misc_feature (18)..(18): 2′ fluoro
  modified_base (19)..(19): gm
  misc_feature (20)..(20): 2′ fluoro
  modified_base (21)..(21): gm
  misc_feature (22)..(22): 2′ fluoro
  modified_base (23)..(23): gm
  misc_feature (24)..(24): 2′ fluoro
  modified_base (25)..(25): um
  misc_feature (26)..(26): 2′ fluoro
  misc_feature (27)..(27): 2′ fluoro
  misc_feature (28)..(28): 2′ fluoro
  modified_base (29)..(29): cm
  misc_feature (30)..(30): 2′ fluoro
  modified_base (31)..(31): cm
  misc_feature (32)..(32): 2′ fluoro
  modified_base (33)..(33): um
  misc_feature (34)..(34): 2′ fluoro
  modified_base (35)..(35): um
  misc_feature (36)..(36): 2′ fluoro
  modified_base (37)..(37): um
  misc_feature (38)..(38): 2′ fluoro, NHC6-linker, trivalent N- acetylgalactosamine
cacagaugugucgaccccgcggggucgacacaucugug

SEQ ID NO: 37 - RNA, 68 nt, Artificial Sequence
  construct Duo / 9 + 12 / st + GN (XD-16858)
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  misc_feature (15)..(19): phosphorothioate linkage
  modified_base (15)..(15): gm
  misc_feature (16)..(16): 2′ fluoro
  modified_base (17)..(17): um
  misc_feature (18)..(18): 2′ fluoro
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine
  modified_base (35)..(35): phospho, gm
  misc_feature (36)..(36): 2′ fluoro
  misc_feature (37)..(37): 2′ methyl
  misc_feature (38)..(38): 2′ fluoro
  modified_base (39)..(39): cm
  misc_feature (40)..(40): 2′ fluoro
  modified_base (41)..(41): um
  misc_feature (42)..(42): 2′ fluoro
  modified_base (43)..(43): um
  misc_feature (44)..(44): 2′ fluoro
  modified_base (45)..(45): gm
  misc_feature (46)..(46): 2′ fluoro
  modified_base (47)..(47): cm
  misc_feature (48)..(48): 2′ fluoro
  misc_feature (49)..(53): phosphorothioate linkage
  modified_base (49)..(49): um
  misc_feature (50)..(50): 2′ fluoro
  modified_base (51)..(51): gm
  misc_feature (52)..(52): 2′ fluoro
  misc_feature (54)..(54): 2′ fluoro
  modified_base (55)..(55): um
  misc_feature (56)..(56): 2′ fluoro
  misc_feature (57)..(57): 2′ fluoro
  misc_feature (58)..(58): 2′ fluoro
  modified_base (59)..(59): um
  misc_feature (60)..(60): 2′ fluoro
  modified_base (61)..(61): um
  misc_feature (62)..(62): 2′ fluoro
  modified_base (63)..(63): cm
  misc_feature (64)..(64): 2′ fluoro
  modified_base (65)..(65): gm
  misc_feature (66)..(66): 2′ fluoro
  modified_base (67)..(67): um
  misc_feature (68)..(68): 2′ fluoro, NHC6-linker, trivalent N- acetylgalactosamine
aaccagaagaagcaggugaaagcccagaagaugcgcaucuucugggcuuuggccugcuucuucugguu

SEQ ID NO: 38 - RNA, 68 nt, Artificial Sequence; identical to SEQ ID NO: 37
  construct Duo / 9 + 12 / unst - GN
  misc_feature (1)..(1): phosphate
  misc_feature (35)..(35): phosphate

SEQ ID NO: 39 - RNA, 102 nt, Artificial Sequence
  construct Trio / 9 + 12 + 15 / st + GN (XD- 16860)
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  modified_base (15)..(15): gm
  misc_feature (15)..(19): phosphorothioate linkage
  misc_feature (16)..(16): 2′ fluoro
  modified_base (17)..(17): um
  misc_feature (18)..(18): 2′ fluoro
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine
  modified_base (35)..(35): phospho, gm
  misc_feature (36)..(36): 2′ fluoro
  misc_feature (37)..(37): 2′ methyl
  misc_feature (38)..(38): 2′ fluoro
  modified_base (39)..(39): cm
  misc_feature (40)..(40): 2′ fluoro
  modified_base (41)..(41): um
  misc_feature (42)..(42): 2′ fluoro
  modified_base (43)..(43): um
  misc_feature (44)..(44): 2′ fluoro
  modified_base (45)..(45): gm
  misc_feature (46)..(46): 2′ fluoro
  modified_base (47)..(47): cm
  misc_feature (48)..(48): 2′ fluoro
  modified_base (49)..(49): um
  misc_feature (49)..(53): phosphorothioate linkage
  misc_feature (50)..(50): 2′ fluoro
  modified_base (51)..(51): gm
  misc_feature (52)..(52): 2′ fluoro
  misc_feature (54)..(54): 2′ fluoro
  modified_base (55)..(55): um
  misc_feature (56)..(56): 2′ fluoro
  misc_feature (57)..(57): 2′ fluoro
  misc_feature (58)..(58): 2′ fluoro
  modified_base (59)..(59): cm
  misc_feature (60)..(60): 2′ fluoro
  misc_feature (61)..(61): 2′ methyl
  misc_feature (62)..(62): 2′ fluoro
  modified_base (63)..(63): gm
  misc_feature (64)..(64): 2′ fluoro
  modified_base (65)..(65): um
  misc_feature (66)..(66): 2′ fluoro
  modified_base (67)..(67): cm
  misc_feature (68)..(68): 2′ fluoro, NHC6-linker, trivalent N- acetylgalactosamine
  modified_base (69)..(69): phospho, um
  misc_feature (70)..(70): 2′ fluoro
  modified_base (71)..(71): um
  misc_feature (72)..(72): 2′ fluoro
  modified_base (73)..(73): cm
  misc_feature (74)..(74): 2′ fluoro
  modified_base (75)..(75): cm
  misc_feature (76)..(76): 2′ fluoro
  misc_feature (77)..(77): 2′ methyl
  misc_feature (78)..(78): 2′ fluoro
  modified_base (79)..(79): gm
  misc_feature (80)..(80): 2′ fluoro
  misc_feature (81)..(81): 2′ methyl
  misc_feature (82)..(82): 2′ fluoro
  modified_base (83)..(83): um
  misc_feature (83)..(87): phosphorothioate linkage
  misc_feature (84)..(84): 2′ fluoro
  modified_base (85)..(85): cm
  misc_feature (86)..(86): 2′ fluoro
  misc_feature (88)..(88): 2′ fluoro
  modified_base (89)..(89): um
  misc_feature (90)..(90): 2′ fluoro
  misc_feature (91)..(91): 2′ fluoro
  misc_feature (92)..(92): 2′ fluoro
  modified_base (93)..(93): um
  misc_feature (94)..(94): 2′ fluoro
  modified_base (95)..(95): um
  misc_feature (96)..(96): 2′ fluoro
  modified_base (97)..(97): cm
  misc_feature (98)..(98): 2′ fluoro
  modified_base (99)..(99): gm
  misc_feature (100)..(100): 2′ fluoro
  modified_base (101)..(101): um
  misc_feature (102)..(102): 2′ fluoro, NHC6-linker, trivalent N- acetylgalactosamine
aaccagaagaagcaggugaaagcccagaagaugcgcaucuucugggcuuuggcauuuccuaggguacauguacccuaggaaauaccacugcuucuucugguu

SEQ ID NO: 40 - RNA, 102 nt, Artificial Sequence; identical to SEQ ID NO: 39
  construct Trio / 9 + 12 + 15 / unst - GN
  misc_feature (1)..(1): phosphate
  misc_feature (35)..(35): phosphate
  misc_feature (69)..(69): phosphate

SEQ ID NO: 41 - RNA, 170 nt, Artificial Sequence
  construct Quinto / 8 + 9 + 12 + 15 + 14 / st + GN (XD-15862)
  misc_feature (1)..(1): phosphate, 2′methyl
  misc_feature (2)..(2): 2′ fluoro
  modified_base (3)..(3): gm
  misc_feature (4)..(4): 2′ fluoro
  misc_feature (5)..(5): 2′ methyl
  misc_feature (6)..(6): 2′ fluoro
  modified_base (7)..(7): cm
  misc_feature (8)..(8): 2′ fluoro
  modified_base (9)..(9): um
  misc_feature (10)..(10): 2′ fluoro
  modified_base (11)..(11): gm
  misc_feature (12)..(12): 2′ fluoro
  misc_feature (13)..(13): 2′ methyl
  misc_feature (14)..(14): 2′ fluoro
  misc_feature (15)..(15): 2′ methyl
  misc_feature (15)..(19): phosphorothioate linkage
  misc_feature (16)..(16): 2′ fluoro
  misc_feature (17)..(17): 2′ methyl
  misc_feature (18)..(18): 2′ fluoro
  misc_feature (20)..(20): 2′ fluoro
  modified_base (21)..(21): um
  misc_feature (22)..(22): 2′ fluoro
  misc_feature (23)..(23): 2′ fluoro
  misc_feature (24)..(24): 2′ fluoro
  modified_base (25)..(25): um
  misc_feature (26)..(26): 2′ fluoro
  modified_base (27)..(27): um
  misc_feature (28)..(28): 2′ fluoro
  modified_base (29)..(29): cm
  misc_feature (30)..(30): 2′ fluoro
  modified_base (31)..(31): gm
  misc_feature (32)..(32): 2′ fluoro
  modified_base (33)..(33): um
  misc_feature (34)..(34): 2′ fluoro, NHC6-linker, trivalent N- acetylgalactosamine
  misc_feature (35)..(35): phospho, 2′ methyl
  misc_feature (36)..(36): 2′ fluoro
  modified_base (37)..(37): cm
  misc_feature (38)..(38): 2′ fluoro
  misc_feature (39)..(39): 2′ methyl
  misc_feature (40)..(40): 2′ fluoro
  misc_feature (41)..(41): 2′ methyl
  misc_feature (42)..(42): 2′ fluoro
  modified_base (43)..(43): gm
  misc_feature (44)..(44): 2′ fluoro
  misc_feature (45)..(45): 2′ methyl
  misc_feature (46)..(46): 2′ fluoro
  modified_base (47)..(47): cm
  misc_feature (48)..(48): 2′ fluoro
  misc_feature (49)..(53): phosphorothioate linkage
  modified_base (49)..(49): gm
  misc_feature (50)..(50): 2′ fluoro
  modified_base (50)..(50): um
  misc_feature (52)..(52): 2′ fluoro
  misc_feature (54)..(54): 2′ fluoro
  misc_feature (55)..(55): 2′ methyl
  misc_feature (56)..(56): 2′ fluoro
  misc_feature (57)..(57): 2′ fluoro
  misc_feature (58)..(58): 2′ fluoro
  modified_base (59)..(59): cm
  misc_feature (60)..(60): 2′ fluoro
  modified_base (61)..(61): gm
  misc_feature (62)..(62): 2′ fluoro
  misc_feature (63)..(63): 2′ methyl
  misc_feature (64)..(64): 2′ fluoro
  misc_feature (65)..(65): 2′ methyl
  misc_feature (66)..(66): 2′ fluoro
  modified_base (67)..(67): gm
  misc_feature (68)..(68): 2′ fluoro, NHC6-linker, trivalent N- acetylgalactosamine
  modified_base (69)..(69): phospho, gm
  misc_feature (70)..(70): 2′ fluoro
  misc_feature (71)..(71): 2′ methyl
  misc_feature (72)..(72): 2′ fluoro
  modified_base (73)..(73): cm
  misc_feature (74)..(74): 2′ fluoro
  modified_base (75)..(75): um
  misc_feature (76)..(76): 2′ fluoro
  modified_base (77)..(77): um
  misc_feature (78)..(78): 2′ fluoro
  modified_base (79)..(79): gm
  misc_feature (80)..(80): 2′ fluoro
  modified_base (81)..(81): cm
  misc_feature (82)..(82): 2′ fluoro
  misc_feature (83)..(87): phosphorothioate linkage
  modified_base (83)..(83): um
  misc_feature (84)..(84): 2′ fluoro
  modified_base (85)..(85): gm
  misc_feature (86)..(86): 2′ fluoro
  misc_feature (88)..(88): 2′ fluoro
  modified_base (89)..(89): um
  misc_feature (90)..(90): 2′ fluoro
  misc_feature (91)..(91): 2′ fluoro
  misc_feature (92)..(92): 2′ fluoro
  modified_base (93)..(93): cm
  misc_feature (94)..(94): 2′ fluoro
  misc_feature (95)..(95): 2′ methyl
  misc_feature (96)..(96): 2′ fluoro
  modified_base (97)..(97): gm
  misc_feature (98)..(98): 2′ fluoro
  modified_base (99)..(99): um
  misc_feature (100)..(100): 2′ fluoro
  modified_base (101)..(101): cm
  misc_feature (102)..(102): 2′ fluoro, NHC6-linker, trivalent N- acetylgalactosamine
  modified_base (103)..(103): phospho, um
  misc_feature (104)..(104): 2′ fluoro
  modified_base (105)..(105): um
  misc_feature (106)..(106): 2′ fluoro
  modified_base (107)..(107): cm
  misc_feature (108)..(108): 2′ fluoro
  modified_base (109)..(109): cm
  misc_feature (110)..(110): 2′ fluoro
  misc_feature (111)..(111): 2′ methyl
  misc_feature (112)..(112): 2′ fluoro
  modified_base (113)..(113): gm
  misc_feature (114)..(114): 2′ fluoro
  misc_feature (115)..(115): 2′ methyl
  misc_feature (116)..(116): 2′ fluoro
  misc_feature (117)..(121): phosphorothioate linkage
  modified_base (117)..(117): um
  misc_feature (118)..(118): 2′ fluoro
  modified_base (119)..(119): cm
  misc_feature (120)..(120): 2′ fluoro
  misc_feature (122)..(122): 2′ fluoro
  modified_base (123)..(123): um
  misc_feature (124)..(124): 2′ fluoro
  misc_feature (125)..(125): 2′ fluoro
  misc_feature (126)..(126): 2′ fluoro
  modified_base (127)..(127): cm
  misc_feature (128)..(128): 2′ fluoro
  modified_base (129)..(129): cm
  misc_feature (130)..(130): 2′ fluoro
  modified_base (131)..(131): um
  misc_feature (132)..(132): 2′ fluoro
  modified_base (133)..(133): um
  misc_feature (134)..(134): 2′ fluoro
  modified_base (135)..(135): um
  misc_feature (136)..(136): 2′ fluoro, NHC6-linker, trivalent N- acetylgalactosamine
  modified_base (137)..(137): phospho, cm
  misc_feature (138)..(138): 2′ fluoro
  modified_base (139)..(139): cm
  misc_feature (140)..(140): 2′ fluoro
  modified_base (141)..(141): gm
  misc_feature (142)..(142): 2′ fluoro
  modified_base (143)..(143): um
  misc_feature (144)..(144): 2′ fluoro
  modified_base (145)..(145): um
  misc_feature (146)..(146): 2′ fluoro
  modified_base (147)..(147): um
  misc_feature (148)..(148): 2′ fluoro
  modified_base (149)..(149): gm
  misc_feature (150)..(150): 2′ fluoro
  misc_feature (151)..(155): phosphorothioate linkage
  modified_base (151)..(151): cm
  misc_feature (152)..(152): 2′ fluoro
  modified_base (153)..(153): cm
  misc_feature (154)..(154): 2′ fluoro
  misc_feature (156)..(156): 2′ fluoro
  modified_base (157)..(157): um
  misc_feature (158)..(158): 2′ fluoro
  misc_feature (159)..(159): 2′ fluoro
  misc_feature (160)..(160): 2′ fluoro
  modified_base (161)..(161): um
  misc_feature (162)..(162): 2′ fluoro
  modified_base (163)..(163): gm
  misc_feature (164)..(164): 2′ fluoro
  modified_base (165)..(165): gm
  misc_feature (166)..(166): 2′ fluoro
  misc_feature (167)..(167): 2′ methyl
  misc_feature (168)..(168): 2′ fluoro
  misc_feature (169)..(169): 2′ methyl
  misc_feature (170)..(170): 2′ fluoro, NHC6-linker, trivalent N- acetylgalactosamine
uuguacccuaggaaauacccugcuucuucugguuaaccagaagaagcaggugaaagcccagaagaugcgcaucuucugggcuuuggcauuuccuaggguacauguacccuaggaaauaccagucgacacaucugugcacagaugugucgaccccguuuccuaggguacaa

SEQ ID NO: 42 - RNA, 204 nt, Artificial Sequence
  Construct Quinto / 8 + 9 + 12 + 15 + 14 / unst - GN
  misc_feature (1)..(1): phosphate
  misc_feature (35)..(35): phosphate
  misc_feature (69)..(69): phosphate, 2′ methyl
  misc_feature (70)..(70): 2′ fluoro
  misc_feature (71)..(71): 2′ methyl
  misc_feature (72)..(72): 2′ fluoro
  misc_feature (73)..(73): cm
  misc_feature (74)..(74): 2′ fluoro
  misc_feature (75)..(75): um
  misc_feature (76)..(76): 2′ fluoro
  misc_feature (77)..(77): um
  misc_feature (78)..(78): 2′ fluoro
  misc_feature (79)..(79): gm
  misc_feature (80)..(80): 2′ fluoro
  misc_feature (81)..(81): cm
  misc_feature (82)..(82): 2′ fluoro
  misc_feature (83)..(83): um
  misc_feature (83)..(87): phosphorothioate linkage
  misc_feature (84)..(84): 2′ fluoro
  misc_feature (85)..(85): gm
  misc_feature (86)..(86): 2′ fluoro
  misc_feature (87)..(87): cm
  misc_feature (88)..(88): 2′ fluoro
  misc_feature (89)..(89): um
  misc_feature (90)..(90): 2′ fluoro
  misc_feature (91)..(91): 2′ fluoro
  misc_feature (92)..(92): 2′ fluoro
  misc_feature (93)..(93): cm
  misc_feature (94)..(94): 2′ fluoro
  misc_feature (95)..(95): 2′ methyl
  misc_feature (96)..(96): 2′ fluoro
  misc_feature (97)..(97): gm
  misc_feature (98)..(98): 2′ fluoro
  misc_feature (99)..(99): um
  misc_feature (100)..(100): 2′ fluoro
  misc_feature (101)..(101): cm
  misc_feature (102)..(102): 2′ fluoro, NHC6-linker, trivalent N- acetylgalactosamine
  misc_feature (103)..(103): phosphate
  misc_feature (137)..(137): phosphate
  misc_feature (171)..(171): phosphate
aaccagaagaagcaggugaaagcccagaagaugcgcaucuucugggcuuuggcauuuccuaggguacagcaucuucugggcuuuggcauuuccuaggguacauguacccuaggaaauaccagucgacacaucugugcacagaugugucgaccccguuuccuaggguacaauuguacccuaggaaauacccugcuucuucugguu

SEQ ID NO: 43 - RNA, 68 nt, Artificial Sequence; identical to SEQ ID NO: 37
  construct XD-17364
  misc_feature (1)..(1): phosphate, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  misc_feature (15)..(19): phosphorothioate linkage
  modified_base (15)..(15): gm
  misc_feature (16)..(16): 2′ fluoro
  modified_base (17)..(17): um
  misc_feature (18)..(18): 2′ fluoro
  misc_feature (19)..(19): 2′ methyl
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine
  misc_feature (35)..(35): phospho, 2′methyl
  misc_feature (36)..(36): 2′ fluoro
  misc_feature (37)..(37): 2′ methyl
  misc_feature (38)..(38): 2′ fluoro
  modified_base (39)..(39): cm
  misc_feature (40)..(40): 2′ fluoro
  modified_base (41)..(41): um
  misc_feature (42)..(42): 2′ fluoro
  modified_base (43)..(43): um
  misc_feature (44)..(44): 2′ fluoro
  modified_base (45)..(45): gm
  misc_feature (46)..(46): 2′ fluoro
  modified_base (47)..(47): cm
  misc_feature (48)..(48): 2′ fluoro
  misc_feature (49)..(53): phosphorothioate linkage
  modified_base (49)..(49): um
  misc_feature (50)..(50): 2′ fluoro
  modified_base (51)..(51): gm
  misc_feature (52)..(52): 2′ fluoro
  modified_base (53)..(53): cm
  misc_feature (54)..(54): 2′ fluoro
  modified_base (55)..(55): um
  misc_feature (56)..(56): 2′ fluoro
  misc_feature (57)..(57): 2′ fluoro
  misc_feature (58)..(58): 2′ fluoro
  modified_base (59)..(59): um
  misc_feature (60)..(60): 2′ fluoro
  modified_base (61)..(61): um
  misc_feature (62)..(62): 2′ fluoro
  modified_base (63)..(63): cm
  misc_feature (64)..(64): 2′ fluoro
  modified_base (65)..(65): gm
  misc_feature (66)..(66): 2′ fluoro
  modified_base (67)..(67): um
  misc_feature (68)..(68): 2′ fluoro, NHC6-linker, trivalent N- acetylgalactosamine

SEQ ID NO: 44 - RNA, 102 nt, Artificial Sequence; identical to SEQ ID NO: 39
  Construct XD-17365
  misc_feature (1)..(1): phospho, 2′ methyl
  misc_feature (2)..(2): 2′ fluoro
  modified_base (3)..(3): cm
  misc_feature (4)..(4): 2′ fluoro
  misc_feature (5)..(5): 2′ methyl
  misc_feature (6)..(6): 2′ fluoro
  misc_feature (7)..(7): 2′ methyl
  misc_feature (8)..(8): 2′ fluoro
  modified_base (9)..(9): gm
  misc_feature (10)..(10): 2′ fluoro
  misc_feature (11)..(11): 2′ methyl
  misc_feature (12)..(12): 2′ fluoro
  modified_base (13)..(13): cm
  misc_feature (14)..(14): 2′ fluoro
  misc_feature (15)..(19): phosphorothioate linkage
  modified_base (15)..(15): gm
  misc_feature (16)..(16): 2′ fluoro
  modified_base (17)..(17): um
  misc_feature (18)..(18): 2′ fluoro
  misc_feature (19)..(19): 2′ methyl
  misc_feature (20)..(20): 2′ fluoro
  misc_feature (21)..(21): 2′ methyl
  misc_feature (22)..(22): 2′ fluoro
  misc_feature (23)..(23): 2′ fluoro
  misc_feature (24)..(24): 2′ fluoro
  modified_base (25)..(25): cm
  misc_feature (26)..(26): 2′ fluoro
  modified_base (27)..(27): gm
  misc_feature (28)..(28): 2′ fluoro
  misc_feature (29)..(29): 2′ methyl
  misc_feature (30)..(30): 2′ fluoro
  misc_feature (31)..(31): 2′ methyl
  misc_feature (32)..(32): 2′ fluoro
  modified_base (33)..(33): gm
  misc_feature (34)..(34): 2′ fluoro, NHC6-linker, trivalent N- acetylgalactosamine
  modified_base (35)..(35): phosphate, gm
  misc_feature (36)..(36): 2′ fluoro
  misc_feature (37)..(37): 2′ methyl
  misc_feature (38)..(38): 2′ fluoro
  modified_base (39)..(39): cm
  misc_feature (40)..(40): 2′ fluoro
  modified_base (41)..(41): um
  misc_feature (42)..(42): 2′ fluoro
  modified_base (43)..(43): um
  misc_feature (44)..(44): 2′ fluoro
  modified_base (45)..(45): gm
  misc_feature (46)..(46): 2′ fluoro
  modified_base (47)..(47): cm
  misc_feature (48)..(48): 2′ fluoro
  misc_feature (49)..(53): phosphorothioate linkage
  modified_base (49)..(49): um
  misc_feature (50)..(50): 2′ fluoro
  modified_base (51)..(51): gm
  misc_feature (52)..(52): 2′ fluoro
  modified_base (53)..(53): cm
  misc_feature (54)..(54): 2′ fluoro
  modified_base (55)..(55): um
  misc_feature (56)..(56): 2′ fluoro
  misc_feature (57)..(57): 2′ fluoro
  misc_feature (58)..(58): 2′ fluoro
  modified_base (59)..(59): cm
  misc_feature (60)..(60): 2′ fluoro
  misc_feature (61)..(61): 2′ methyl
  misc_feature (62)..(62): 2′ fluoro
  modified_base (63)..(63): gm
  misc_feature (64)..(64): 2′ fluoro
  modified_base (65)..(65): um
  misc_feature (66)..(66): 2′ fluoro
  modified_base (67)..(67): cm
  misc_feature (68)..(68): 2′ fluoro, NHC6-linker, trivalent N- acetylgalactosamine
  modified_base (69)..(69): phospho, um
  misc_feature (70)..(70): 2′ fluoro
  modified_base (71)..(71): um
  misc_feature (72)..(72): 2′ fluoro
  modified_base (73)..(73): cm
  misc_feature (74)..(74): 2′ fluoro
  modified_base (75)..(75): cm
  misc_feature (76)..(76): 2′ fluoro
  misc_feature (77)..(77): 2′ methyl
  misc_feature (78)..(78): 2′ fluoro
  modified_base (79)..(79): gm
  misc_feature (80)..(80): 2′ fluoro
  misc_feature (81)..(81): 2′ methyl
  misc_feature (82)..(82): 2′ fluoro
  misc_feature (83)..(87): phosphorothioate linkage
  modified_base (83)..(83): um
  misc_feature (84)..(84): 2′ fluoro
  modified_base (85)..(85): cm
  misc_feature (86)..(86): 2′ fluoro
  misc_feature (87)..(87): 2′ methyl
  misc_feature (88)..(88): 2′ fluoro
  modified_base (89)..(89): um
  misc_feature (90)..(90): 2′ fluoro
  misc_feature (91)..(91): 2′ fluoro
  misc_feature (92)..(92): 2′ fluoro
  modified_base (93)..(93): um
  misc_feature (94)..(94): 2′ fluoro
  modified_base (95)..(95): um
  misc_feature (96)..(96): 2′ fluoro
  modified_base (97)..(97): cm
  misc_feature (98)..(98): 2′ fluoro
  modified_base (99)..(99): gm
  misc_feature (100)..(100): 2′ fluoro
  modified_base (101)..(101): um
  misc_feature (102)..(102): 2′ fluoro, NHC6-linker, trivalent N- acetylgalactosamine

SEQ ID NO: 45 - RNA, 170 nt, Artificial Sequence
  construct XD-17366
  misc_feature (1)..(1): phosphate, 2′ methyl
  misc_feature (2)..(2): 3′ fluoro
  modified_base (3)..(3): cm
  misc_feature (4)..(4): 3′ fluoro
  misc_feature (5)..(5): 2′ methyl
  misc_feature (6)..(6): 3′ fluoro
  misc_feature (7)..(7): 2′ methyl
  misc_feature (8)..(8): 3′ fluoro
  modified_base (9)..(9): gm
  misc_feature (10)..(10): 3′ fluoro
  misc_feature (11)..(11): 2′ methyl
  misc_feature (12)..(12): 3′ fluoro
  modified_base (13)..(13): cm
  misc_feature (14)..(14): 3′ fluoro
  modified_base (15)..(15): gm
  misc_feature (16)..(16): 3′ fluoro
  modified_base (17)..(17): um
  misc_feature (18)..(18): 3′ fluoro
  misc_feature (19)..(19): 2′ methyl
  misc_feature (20)..(20): 3′ fluoro
  misc_feature (21)..(21): 2′ methyl
  misc_feature (22)..(22): 3′ fluoro
  misc_feature (23)..(23): 3′ fluoro
  misc_feature (24)..(24): 3′ fluoro
  modified_base (25)..(25): cm
  misc_feature (26)..(26): 3′ fluoro
  modified_base (27)..(27): gm
  misc_feature (28)..(28): 3′ fluoro
  misc_feature (29)..(29): 2′ methyl
  misc_feature (30)..(30): 3′ fluoro
  misc_feature (31)..(31): 2′ methyl
  misc_feature (32)..(32): 3′ fluoro
  modified_base (33)..(33): gm
  misc_feature (34)..(34): 3′ fluoro, NHC6-linker, trivalent N- acetylgalactosamine
  misc_feature (35)..(35): phosphate, 2′ methyl
  misc_feature (36)..(36): 3′ fluoro
  misc_feature (37)..(37): 2′ methyl
  misc_feature (38)..(38): 3′ fluoro
  modified_base (39)..(39): cm
  misc_feature (40)..(40): 3′ fluoro
  modified_base (41)..(41): um
  misc_feature (42)..(42): 3′ fluoro
  modified_base (43)..(43): um
  misc_feature (44)..(44): 3′ fluoro
  modified_base (45)..(45): gm
  misc_feature (46)..(46): 3′ fluoro
  modified_base (47)..(47): cm
  misc_feature (48)..(48): 3′ fluoro
  misc_feature (49)..(53): phosphorothioate linkage
  modified_base (49)..(49): um
  misc_feature (50)..(50): 3′ fluoro
  modified_base (51)..(51): gm
  misc_feature (52)..(52): 3′ fluoro
  modified_base (53)..(53): cm
  misc_feature (54)..(54): 3′ fluoro
  modified_base (55)..(55): um
  misc_feature (56)..(56): 3′ fluoro
  misc_feature (57)..(57): 3′ fluoro
  misc_feature (58)..(58): 3′ fluoro
  modified_base (59)..(59): cm
  misc_feature (60)..(60): 3′ fluoro
  misc_feature (61)..(61): 2′ methyl
  misc_feature (62)..(62): 3′ fluoro
  modified_base (63)..(63): gm
  misc_feature (64)..(64): 3′ fluoro
  modified_base (65)..(65): um
  misc_feature (66)..(66): 3′ fluoro
  modified_base (67)..(67): cm
  misc_feature (68)..(68): 3′ fluoro, NHC6-linker, trivalent N- acetylgalactosamine
  misc_feature (69)..(69): phosphate, 2′ methyl
  misc_feature (70)..(70): 3′ fluoro
  modified_base (71)..(71): um
  misc_feature (72)..(72): 3′ fluoro
  modified_base (73)..(73): cm
  misc_feature (74)..(74): 3′ fluoro
  modified_base (75)..(75): cm
  misc_feature (76)..(76): 3′ fluoro
  misc_feature (77)..(77): 2′ methyl
  misc_feature (78)..(78): 3′ fluoro
  modified_base (79)..(79): gm
  misc_feature (80)..(80): 3′ fluoro
  misc_feature (81)..(81): 2′ methyl
  misc_feature (82)..(82): 3′ fluoro
  misc_feature (83)..(87): phosphorothioate linkage
  modified_base (83)..(83): um
  misc_feature (84)..(84): 3′ fluoro
  modified_base (85)..(85): cm
  misc_feature (86)..(86): 3′ fluoro
  misc_feature (87)..(87): 2′ methyl
  misc_feature (88)..(88): 3′ fluoro
  modified_base (89)..(89): um
  misc_feature (90)..(90): 3′ fluoro
  misc_feature (91)..(91): 3′ fluoro
  misc_feature (92)..(92): 3′ fluoro
  modified_base (93)..(93): cm
  misc_feature (94)..(94): 3′ fluoro
  modified_base (95)..(95): cm
  misc_feature (96)..(96): 3′ fluoro
  modified_base (97)..(97): um
  misc_feature (98)..(98): 3′ fluoro
  modified_base (99)..(99): um
  misc_feature (100)..(100): 3′ fluoro
  modified_base (101)..(101): um
  misc_feature (102)..(102): 3′ fluoro, NHC6-linker, trivalent N- acetylgalactosamine
  misc_feature (103)..(103): phosphate, 2′ methyl
  misc_feature (104)..(104): 3′ fluoro
  modified_base (105)..(105): cm
  misc_feature (106)..(106): 3′ fluoro
  modified_base (107)..(107): gm
  misc_feature (108)..(108): 3′ fluoro
  modified_base (109)..(109): um
  misc_feature (110)..(110): 3′ fluoro
  modified_base (111)..(111): um
  misc_feature (112)..(112): 3′ fluoro
  modified_base (113)..(113): um
  misc_feature (114)..(114): 3′ fluoro
  modified_base (115)..(115): gm
  misc_feature (116)..(116): 3′ fluoro
  misc_feature (117)..(121): phosphorothioate linkage
  modified_base (117)..(117): cm
  misc_feature (118)..(118): 3′ fluoro
  modified_base (119)..(119): cm
  misc_feature (120)..(120): 3′ fluoro
  modified_base (121)..(121): gm
  misc_feature (122)..(122): 3′ fluoro
  modified_base (123)..(123): um
  misc_feature (124)..(124): 3′ fluoro
  misc_feature (125)..(125): 3′ fluoro
  misc_feature (126)..(126): 3′ fluoro
  modified_base (127)..(127): um
  misc_feature (128)..(128): 3′ fluoro
  modified_base (129)..(129): gm
  misc_feature (130)..(130): 3′ fluoro
  modified_base (131)..(131): gm
  misc_feature (132)..(132): 3′ fluoro
  misc_feature (133)..(133): 2′ methyl
  misc_feature (134)..(134): 3′ fluoro
  misc_feature (135)..(135): 2′ methyl
  misc_feature (136)..(136): 3′ fluoro, NHC6-linker, trivalent N- acetylgalactosamine
  misc_feature (137)..(137): phosphate, 2′ methyl
  misc_feature (138)..(138): 3′ fluoro
  modified_base (139)..(139): gm
  misc_feature (140)..(140): 3′ fluoro
  misc_feature (141)..(141): 2′ methyl
  misc_feature (142)..(142): 3′ fluoro
  modified_base (143)..(143): cm
  misc_feature (144)..(144): 3′ fluoro
  modified_base (145)..(145): um
  misc_feature (146)..(146): 3′ fluoro
  modified_base (147)..(147): gm
  misc_feature (148)..(148): 3′ fluoro
  misc_feature (149)..(149): 2′ methyl
  misc_feature (150)..(150): 3′ fluoro
  misc_feature (151)..(151): 2′ methyl
  misc_feature (151)..(155): phosphorothioate linkage
  misc_feature (152)..(152): 3′ fluoro
  misc_feature (153)..(153): 2′ methyl
  misc_feature (154)..(154): 3′ fluoro
  modified_base (155)..(155): cm
  misc_feature (156)..(156): 3′ fluoro
  modified_base (157)..(157): um
  misc_feature (158)..(158): 3′ fluoro
  misc_feature (159)..(159): 3′ fluoro
  misc_feature (160)..(160): 3′ fluoro
  modified_base (161)..(161): um
  misc_feature (162)..(162): 3′ fluoro
  modified_base (163)..(163): um
  misc_feature (164)..(164): 3′ fluoro
  modified_base (165)..(165): cm
  misc_feature (166)..(166): 3′ fluoro
  modified_base (167)..(167): gm
  misc_feature (168)..(168): 3′ fluoro
  modified_base (169)..(169): um
  misc_feature (170)..(170): 3′ fluoro, NHC6-linker, trivalent N- acetylgalactosamine
aaccagaagaagcaggugaaagcccagaagaugcgcaucuucugggcuuuggcauuuccuaggguacauguacccuaggaaauaccagucgacacaucugugcacagaugugucgaccccguuuccuaggguacaauuguacccuaggaaauacccugcuucuucugguu

(19) United States
(12) Patent Application Publication
(10) Pub. No.: US 2021/0371861 A1
(43) Pub. Date: Dec. 2, 2021

(54) Multi-Targeting Nucleic Acid Constructs Composed Of Multiple Oligonucleotides That Modulate Gene Expression Through Complimentary Interactions With Targets

(71) Applicant: Sirnaomics, Inc., Gatithersburg, MD (US)

(72) Inventor: Dmitry SAMARSKY, Gaithersburg, MD (US)

(21) Appl. No.: 17/215,964

(22) Filed: Mar. 29, 2021

Related U.S. Application Data
(63) Continuation of application No. PCT/IB2019/058221, filed on Sep. 27, 2019.
(60) Provisional application No. 62/738,222, filed on Sep. 28, 2018.

(51) Int. Cl. C12N 15/113; A61K 47/54

(52) U.S. Cl. CPC C12N 15/113; A61K 47/549; C12N 2330/30; C12N 2320/30; C12N 2310/322; C12N 2310/51; C12N 2310/313; C12N 2310/315; C12N 2310/321

(57) ABSTRACT

The present invention provides a multi-targeting nucleic acid construct comprising at least: (a) a first nucleic acid portion that is at least partially complementary to at least a first portion of RNA transcribed from a target gene; (b) a second nucleic acid portion that is at least partially complementary to at least a second portion of RNA transcribed from a target gene, which target gene may be the same or different to the target gene defined in (a); (c) a third nucleic acid portion that is at least partially complementary to the first nucleic acid portion of (a), so as to form a first nucleic acid duplex region therewith; (d) a fourth nucleic acid portion that is at least partially complementary to said second nucleic acid portion of (b), so as to form a second nucleic acid duplex region therewith. The construct is designed so that subsequent to in vivo administration the construct disassembles to yield at least first and second discrete nucleic acid targeting molecules that respectively target RNA transcribed from the target genes of (a) and (b). Typically, the first nucleic acid targeting molecule is capable of modulating expression of the target gene of (a), and comprises, or is derived from, at least the first nucleic acid portion of (a). Typically, the second nucleic acid targeting molecule is capable of modulating expression of said target gene of (b), and comprises, or is derived from, the second nucleic acid portion of (b).

### CROSS REFERENCE TO RELATED APPLICATIONS

This application is a continuation of International Application No. PCT/IB2019/058221, filed on Sep. 27, 2019, which claims the benefit of U.S. Provisional Application No. 62/738,222, filed on Sep. 28, 2018, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Apr. 16, 2021, is named 4690_0043C_SL.txt and is 117,499 bytes in size.

The present invention is in the technical field of nanotechnology and/or modulation, which is down-regulation or up-regulation, of gene expression in eukaryotic organisms. Such modulation of gene expression in eukaryotic organisms uses complementary oligonucleotides according to the present invention, typically assembled in nano-structures. More particularly, the present invention is in the technical field of modulation of gene expression in eukaryotic organisms using complementary oligonucleotides assembled in nano-structures to study gene function, treat diseases and/or other applications, including, but not limited to cosmetics and/or agriculture.

The present invention takes advantage of structural flexibility of oligonucleotides to form nano-structures and the ability of antisense oligonucleotide (ASO) and RNA interference (RNAi) molecules, here combined as complimentary oligonucleotides (in this document used as “CON”), to modulate gene expression. Therefore, it integrates components and knowledge belonging to two technological fields—nanotechnology and CON technology.

According to current definition by the US government sponsored National Nanotechnology Initiative, “nanotechnology is the understanding and control of matter at the nanoscale, at dimensions between approximately 1 and 100 nm, where unique phenomena enable novel applications” [http://nano.gov]. Nanotechnology engages research from diverse sciences, including organic chemistry, materials, semiconductor physics, molecular biology, engineering and other, with the vision of creating new nano-materials and molecular devices with numerous applications for electronics, IT, medicine, energy, and everyday life.

The CON technology engages ability of artificially created oligonucleotide-based molecules to interact through complimentary interactions with and change the properties of biological oligonucleotide targets. In its most widely used application, CON molecules are designed to intra-cellularly bind and inactivate protein-coding (i.e. mRNA) or non-coding (e.g. miRNA, IncRNA) molecules typically resulting in silencing of the corresponding genes. Deciphering the silencing results may allow understanding of the function of the genes, and thus be used in functional genomics. Down-regulation of malignant genes or up-regulation of deficient genes with CON molecules in animals, including humans may also allow developing new therapeutic drugs. CON molecules promise utility in other fields as well, including cosmetics, bio-production, agro-biology, and everyday life.

The CON molecules have found broad and diverse application as research tools and offer strong potential to become a third major therapeutic modality, besides small molecules and biologics. Indeed, RNAi-based reagents are routinely used in thousands of research and development laboratories worldwide to study gene functions in eukaryotes, and are finding their ways to clinical trials as gene expression modulating drug candidates. ASO-based technology has been explored for a longer time, in particular as therapeutics, and the first potentially commercially viable drug (Mipomersen, Isis Pharmaceuticals) has been recently approved for the market in the United States. The first RNAi drug, Patisiran, has been approved by the FDA in 2018.

Despite these obvious and impressive successes, CON technology still has much room for improvement. Indeed, conventional RNAi reagents may reveal one or more of the following deficiencies: 1) cumbersome synthesis process and relatively high manufacturing cost (in case of RNAi, for example, requiring making and annealing two oligonucleotides, while only one serving as an active agent); 2) high sensitivity to various endo- and exo-nucleases, and, hence, low stability in any biologic fluids; 3) suboptimal hit rate and efficacy (even with current improved algorithms, there is no guarantee that individual molecules would produce effective target knockdown); 4) non-specific activity and side effects (in case of RNAi, originated from passenger strand, miRNA-associated activity, and in case of both RNAi and ASO originating in particular chemistries and sequences); 5) difficulty of delivering in cell culture, and especially in vivo.

The present invention provides novel compositions and methods, which include specially designed self-assembling nano-structures composed of multiple oligonucleotides and able to modulate gene expression through complimentary interactions with the targets. The present invention offers to address and improve shortcomings associated with the complementary oligonucleotide technologies (e.g. antisense and RNAi technologies), such as high cost of production, suboptimal efficacy and specificity, low stability of molecules in biological fluids and inside the cells, and difficulty of delivery in cell culture and in vivo.

According to the present invention, therefore, there is provided a nucleic acid construct comprising at least:
(a) a first nucleic acid portion that is at least partially complementary to at least a first portion of RNA transcribed from a target gene;
(b) a second nucleic acid portion that is at least partially complementary to at least a second portion of RNA transcribed from a target gene, which target gene may be the same or different to the target gene defined in (a);
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith;
wherein the construct is designed such that subsequent to in vivo administration the construct disassembles to yield at least first and second discrete nucleic acid targeting molecules that respectively target the RNA portions transcribed from the target genes of (a) and (b);
whereby (i) the first nucleic acid targeting molecule is capable of modulating expression of the target gene of (a), and comprises, or is derived from, at least the first nucleic acid portion of (a), and (ii) the second nucleic acid targeting molecule is capable of modulating expression of the target gene of (b), and comprises, or is derived from, the second nucleic acid portion of (b).

In a first embodiment, a construct according to the present invention is designed to disassemble such that the first and second discrete nucleic acid targeting molecules are respectively processed by independent RNAi-induced silencing complexes.

In a second embodiment, a construct according to the present invention further comprises labile functionality such that subsequent to in vivo administration the construct is cleaved so as to yield the at least first and second discrete nucleic acid targeting molecules. Typically, the labile functionality comprises one or more unmodified nucleotides that can represent one or more cleavage positions within the construct, whereby subsequent to in vivo administration the construct is cleaved at the one or more cleavage positions so as to yield the at least first and second discrete nucleic acid targeting molecules.

According to the above described second embodiment, the cleavage positions can be respectively located within the construct so that subsequent to cleavage the first discrete nucleic acid targeting molecule comprises, or is derived from, the first nucleic acid duplex region, and the second discrete nucleic acid targeting molecule comprises, or is derived from, the second nucleic acid duplex region.

The primary structure of a construct according to the present invention is suitably such that the first nucleic acid portion of (a) is directly or indirectly linked to the fourth nucleic acid portion of (d) as a primary structure. When such a construct according to the present invention is a dual targeting construct, typically the second nucleic acid portion of (b) is directly or indirectly linked to the third nucleic acid portion of (c) as a primary structure.

A construct according to the present invention can be dual targeting. Alternatively, the construct can target more than two portions of RNA transcribed from one or more target genes, and in such cases the construct can further comprise 1 to 8 additional nucleic acid portions that are respectively at least partially complementary to an additional 1 to 8 portions of RNA transcribed from one or more target genes, which target genes may be the same or different to each other, and/or the same or different to the target genes as hereinbefore defined in (a) and/or (b), and wherein each of the 1 to 8 additional nucleic acid portions respectively form additional duplex regions with respective passenger nucleic acid portions that are respectively at least partially complementary therewith. In such constructs, the second nucleic acid portion of (b), and the 1 to 8 additional nucleic acid portions, are directly or indirectly linked to selected passenger nucleic acid portions as respective primary structures.

As hereinbefore described, there may be direct or indirect linking between respective positions of a construct according to the present invention. Such direct or indirect linking represents either (i) an internucleotide nick, (ii) an internucleotide bond, or (iii) a nucleic acid linker portion of 1 to 10 nucleotides, wherein in the case of (i) there exists some complementarity between the first nucleic acid portion of (a) and the second nucleic acid portion of (b), or the third nucleic acid portion of (c) and the fourth nucleic acid portion of (d).

A construct according to the present invention can be represented by the following schematic structure:

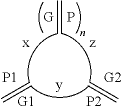

wherein
G1 represents the first nucleic acid portion of (a);
G2 represents the second nucleic acid portion of (b);
P1 represents the third nucleic acid portion of (c);
P2 represents the fourth nucleic acid portion of (d);
G represents the 1 to 8 additional nucleic acid portions that are respectively at least partially complementary to an additional 1 to 8 portions of RNA transcribed from one or more target genes;
P represents the passenger nucleic acid portions that are respectively at least partially complementary with the 1 to 8 additional nucleic acid portions and forming the duplex regions therewith;
each of G1, G2, P1, P2 can each respectively include the same or different numbers of nucleotides;
n is an integer selected between 0 to 8;
wherein there is present one or more adjacent and/or non-adjacent cleavage positions, that at least allows disassembly of at least G1 from P2, and/or at least G2 from P1, and when n is 1 to 8 there is also present one or more adjacent and/or non-adjacent cleavage positions that allows disassembly of at least G2 from an adjacent P, and/or at least P1 from an adjacent G;
each of x, y, z either represent (i) an internucleotide nick, (ii) a internucleotide bond, or (iii) a nucleic acid linker portion of 1 to 10 nucleotides;
wherein when n is 0, and x, y, z represent an internucleotide nick between G1 and P2, and P1 and G2 respectively, then there exists some complementarity between either G1 and G2, or P1 and P2.

A nucleic acid linker portion that can be present in a construct according to the present invention as hereinbefore described is typically single stranded.

A construct according to the present invention preferably further comprises one or more ligands, typically conjugated to the third nucleic acid portion of (c), and/or the fourth nucleic acid portion of (d), and/or the passenger nucleic acid portions as hereinbefore described.

The first nucleic acid portion of (a), and/or the second nucleic acid portion of (b), and/or the third nucleic acid portion of (c), and/or the fourth nucleic acid portion of (d), and/or the 1 to 8 additional nucleic acid portions and/or the passenger nucleic acid portions respectively have a 5′ to 3′ directionality thereby defining 5′ and 3′ regions thereof, and wherein the one or more ligands are conjugated at the 3 ‘ region, or at one or more regions intermediate of the 5’ and 3′ regions, of any of (i) the third nucleic acid portion of (c), and/or (ii) the fourth nucleic acid portion of (d), and/or (iii) the passenger nucleic acid portions.

The one or more ligands are any cell directing moiety, such as lipids, carbohydrates, aptamers, vitamins and/or peptides that bind cellular membrane or a specific target on cellular surface, preferably one or more carbohydrates, that can suitably be a monosaccharide, disaccharide, trisaccharide, tetrasaccharide, oligosaccharide or polysaccharide. Still more preferably, the one or more carbohydrates comprise one or more galactose moieties, one or more lactose moieties, one or more N-Acetyl-Galactosamine moieties, and/or one or more mannose moieties.

Particularly preferred is wherein the one or more carbohydrates comprise one or more N-Acetyl-Galactosamine moieties, in particular two or three N-Acetyl-Galactosamine moieties, that can be attached in a linear configuration, or in a branched configuration. A branched configuration can be desirable, wherein one or more ligands are attached as a biantennary or triantennary configuration. Alternatively, the ligand configuration can be based on single ligands at different positions.

A construct according to the present invention can have portions of selected length corresponding to the RNA sequence to be targeted. For example, the first nucleic acid portion of (a), and/or the second nucleic acid portion of (b), and/or the third nucleic acid portion of (c), and/or the fourth nucleic acid portion of (d), can be respectively 7 to 20 nucleotides in length, preferably 10 to 18 nucleotides in length, more preferably about 15 nucleotides in length. Typically, when a nucleic acid linker portion is present, this may be 1 to 8 nucleotides in length, preferably 2 to 6 nucleotides in length, more preferably about 4 nucleotides in length.

A construct according to the present invention can preferably further comprise one or more phosphorothioate or phosphorodithioate internucleotide linkages, such as 1 to 15 phosphorothioate or phosphorodithioate internucleotide linkages. Such one or more phosphorothioate or phosphorodithioate internucleotide linkages are typically present at one or more of the 5′ and/or 3′ regions of the first nucleic acid portion of (a), and/or the second nucleic acid portion of (b), and/or the third nucleic acid portion of (c), and/or the fourth nucleic acid portion of (d), and/or 1 to 8 additional nucleic acid portions, and/or the passenger nucleic acid portions.

A construct according to the present invention can also comprise phosphorothioate or phosphorodithioate internucleotide linkages between at least two adjacent nucleotides of the nucleic acid linker portion, and more preferably can comprise a phosphorothioate or phosphorodithioate internucleotide linkage between each adjacent nucleotide that is present in the nucleic acid linker portion.

A construct according to the present invention can suitably comprise a phosphorothioate or phosphorodithioate internucleotide linkage linking:

the first nucleic acid portion of (a) to a nucleic acid linker portion; and/or
the second nucleic acid portion of (b) to a nucleic acid linker portion and/or
the third nucleic acid portion of (c) to a nucleic acid linker portion; and/or
the fourth nucleic acid portion of (d) to a nucleic acid linker portion; and/or
the 1 to 8 additional nucleic acid portions to a nucleic acid linker portion; and/or
the passenger nucleic acid portions to a nucleic acid linker portion.

Typically, a construct according to the present invention is modified. For example, at least one nucleotide of at least one of the following is modified:
the first nucleic acid portion of (a); and/or
the second nucleic acid portion of (b); and/or
the third nucleic acid portion of (c); and/or
the fourth nucleic acid portion of (d); and/or
the 1 to 8 additional nucleic acid portions; and/or
the passenger nucleic acid portions; and/or
the nucleic acid linker portion.

Typically, the modification can be such that one or more of the odd numbered nucleotides starting from the 5′ region of one of the following are modified, and/or wherein one or more of the even numbered nucleotides starting from the 5′ region of one of the following are modified, wherein typically the modification of the even numbered nucleotides is a second modification that is different from the modification of odd numbered nucleotides:
the first nucleic acid portion of (a); and/or
the second nucleic acid portion of (b); and/or
the third nucleic acid portion of (c); and/or
the fourth nucleic acid portion of (d); and/or
the 1 to 8 additional nucleic acid portions; and/or
the passenger nucleic acid portions.

Still further, the modification may be such that one or more of the odd numbered nucleotides starting from the 3′ region of the third nucleic acid portion of (c) are modified by a modification that is different from the modification of odd numbered nucleotides starting from the 5′ region of the first nucleic acid portion of (a); and/or
wherein one or more of the odd numbered nucleotides starting from the 3′ region of the fourth nucleic acid portion of (d) are modified by a modification that is different from the modification of odd numbered nucleotides starting from the 5′ region of the second nucleic acid portion of (b); and/or
wherein one or more of the odd numbered nucleotides starting from the 3′ region of the passenger nucleic acid portions are modified by a modification that is different from the modification of odd numbered nucleotides starting from the 5′ region of the 1 to 8 additional nucleic acid portions; and/or
wherein one or more of the nucleotides of a nucleic acid linker portion are modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions.

Still further, the modification can be such that one or more of the even numbered nucleotides starting from the 3′ region of: (i) the third nucleic acid portion of (c), and/or (ii) the fourth nucleic acid portion of (d), and/or (iii) the passenger nucleic acid portions, are modified by a modification that is different from the modification of odd numbered nucleotides starting from the 3′ region of these respective portions.

Still further, the modification can be such that at least one or more of the modified even numbered nucleotides of (i) the first nucleic acid portion of (a), and/or (ii) the second nucleic acid portion of (b), and/or (iii) the 1 to 8 additional nucleic acid portions, is adjacent to at least one or more differently modified odd numbered nucleotides of these respective portions.

Still further, the modification can be such that at least one or more of the modified even numbered nucleotides of (i) the third nucleic acid portion of (c), and/or (ii) the fourth nucleic acid portion of (d), and/or (iii) the passenger nucleic acid portions, is adjacent to at least one or more differently modified odd numbered nucleotides of these respective portions.

Still further, the modification can be such that a plurality of adjacent nucleotides of (i) the first nucleic acid portion of (a), and/or (ii) the second nucleic acid portion of (b), and/or (iii) the 1 to 8 additional nucleic acid portions, are modified by a common modification.

Still further, the modification can be such that a plurality of adjacent nucleotides of (i) the third nucleic acid portion of (c), and/or (ii) the fourth nucleic acid portion of (d), and/or (iii) the passenger nucleic acid portions, are modified by a common modification, which can be 2 to 4 adjacent nucleotides, preferably 3 or 4 adjacent nucleotides. Typically, the plurality of adjacent commonly modified nucleotides are located in the 5′ region of (i) the third nucleic acid portion of (c), and/or (ii) the fourth nucleic acid portion of (d), and/or (iii) the passenger nucleic acid portions and/or can be located in the nucleic acid linker portion.

Still further, the modification can be such that the one or more of the modified nucleotides of first nucleic acid portion of (a) do not have a common modification present in the corresponding nucleotide of the third nucleic acid portion of (c) of the first duplex region; and/or one or more of the modified nucleotides of second nucleic acid portion of (b) do not have a common modification present in the corresponding nucleotide of the fourth nucleic acid portion of (d) of the second duplex region; and/or one or more of the modified nucleotides of the 1 to 8 additional nucleic acid portions do not have a common modification present in the corresponding nucleotide of the corresponding passenger nucleic acid portions of the respective duplex regions.

Still further, the modification can be such that the one or more of the modified nucleotides of the first nucleic acid portion of (a) are shifted by at least one nucleotide relative to a commonly modified nucleotide of the third nucleic acid portion of (c); and/or one or more of the modified nucleotides of the second nucleic acid portion of (b) are shifted by at least one nucleotide relative to a commonly modified nucleotide of the fourth nucleic acid portion of (d); and/or one or more of the modified nucleotides of the 1 to 8 additional nucleic acid portions are shifted by at least one nucleotide relative to a commonly modified nucleotide of the passenger nucleic acid portions.

Typically, the modification and/or modifications are each and individually sugar, backbone or base modifications, and are suitably selected from the group consisting of 3′-terminal deoxy-thymine, 2′-O-methyl, a 2′-deoxy-modification, a 2′-amino-modification, a 2′-alkyl-modification, a morpholino modification, a phosphoramidate modification, phosphorothioate or phosphorodithioate group modification, a 5′ phosphate or 5′ phosphate mimic modification and a cholesteryl derivative or a dodecanoic acid bisdecylamide group modification. The modification can be any one of a locked nucleotide, an abasic nucleotide or a non-natural base comprising nucleotide.

Preferably, at least one modification is a 2′-O-methyl modification in a ribose moiety.

Preferably, at least one modification is a 2′-F modification in a ribose moiety.

Still further, the modification can be such that the nucleotides at any of positions 2 and 14 downstream from the first nucleotide of the 5′ region of (i) the first nucleic acid portion of (a); and/or (ii) the second nucleic acid portion of (b); and/or (iii) the 1 to 8 additional nucleic acid portions; do not contain 2′-O-methyl modifications in ribose moieties.

Still further, the modification can be such that the nucleotides of (i) the third nucleic acid portion of (c); and or (ii) the fourth nucleic acid portion of (d); and/or (iii) the passenger nucleic acid portions; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions; do not contain 2′-O-methyl modifications in ribose moieties.

Still further, the modification can be such that the nucleotides at any of positions 2 and 14 downstream from the first of (i) the first nucleic acid portion of (a); and/or (ii) the second nucleic acid portion of (b); and/or (iii) the 1 to 8 additional nucleic acid portions; contain 2′-F modifications in ribose moieties.

Still further, the modification can be such that the nucleotides of (i) the third nucleic acid portion of (c); and or (ii) the fourth nucleic acid portion of (d); and/or (iii) the passenger nucleic acid portions; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions; contain 2′-F modifications in ribose moieties.

A construct according to the present invention preferably comprises one or more unmodified nucleotides. These one or more unmodified nucleotides can replace any modified nucleotide as hereinbefore described. Preferably the one or more, preferably one, unmodified nucleotide represents any of the nucleotides of the nucleic acid linker portion as hereinbefore described, preferably the nucleotide of the nucleic acid linker portion that is adjacent to (i) the third nucleic acid portion of (c); and or (ii) the fourth nucleic acid portion of (d); and/or (iii) the passenger nucleic acid portions.

Methyl modification can be a preferred chemical modification in a gene modulating molecule, as it represents a naturally occurring nucleotide modification. Preferably therefore, a conjugate according to the present invention is such that all nucleotides other than
the unmodified nucleotides; and/or
the nucleotides at any of positions 2 and 14 downstream from the first nucleotide of the 5′ region of (i) the first nucleic acid portion of (a); and/or (ii) the second nucleic acid portion of (b); and/or (iii) the 1 to 8 additional nucleic acid portions; and/or
the nucleotides of (i) the third nucleic acid portion of (c); and or (ii) the fourth nucleic acid portion of (d); and/or (iii) the passenger nucleic acid portions; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions;
contain 2′-O-methyl modifications in ribose moieties.

A construct according to the present invention can also comprise at least one vinylphosphonate modification, such as at least one vinylphosphonate modification in the 5′ region of (i) the first nucleic acid portion of (a); and/or (ii) the second nucleic acid portion of (b); and/or (iii) the 1 to 8 additional nucleic acid portions.

Still further in a construct according to the present invention, one or more nucleotides of
the first nucleic acid portion of (a); and/or
the second nucleic acid portion of (b); and/or
the third nucleic acid portion of (c); and/or
the fourth nucleic acid portion of (d); and/or
the 1 to 8 additional nucleic acid portions; and/or
the passenger nucleic acid portions;
is an inverted nucleotide and is attached to the adjacent nucleotide via the 3′ carbon of the nucleotide and the 3′ carbon of the adjacent nucleotide, and/or is an inverted nucleotide and is attached to the adjacent nucleotide via the 5′ carbon of the nucleotide and the 5′ carbon of the adjacent nucleotide.

Typically, such an inverted nucleotide is attached to the adjacent nucleotide via a phosphate group by way of a phosphodiester linkage; or is attached to the adjacent nucleotide via a phosphorothioate group; or is attached to the adjacent nucleotide via a phosphorodithioate group.

A construct according to the present invention can be blunt ended. Alternatively, in a conjugate according to the present invention:
the first nucleic acid portion of (a); and/or
the second nucleic acid portion of (b); and/or
the third nucleic acid portion of (c); and/or
the fourth nucleic acid portion of (d); and/or
the 1 to 8 additional nucleic acid portions; and/or
the passenger nucleic acid portions;
has an overhang.

A construct according to the present invention is typically directed against target RNA that is selected from at least one of: mRNA, IncRNA, and/or other RNA molecules.

The present invention also provides a composition comprising a construct as hereinbefore described, and a physiologically acceptable excipient.

The present invention also provides a construct as hereinbefore described, for use in the treatment of a disease or disorder.

The present invention also provides use of a construct as hereinbefore described, in the manufacture of a medicament for treating a disease or disorder.

The present invention also provides a method of treating a disease or disorder comprising administration of a construct as hereinbefore described, to an individual in need of treatment.

Preferably in such a method, the construct is administered subcutaneously or intravenously to the individual. Furthermore, in such a method, subsequent to in vivo administration the construct disassembles to yield at least first and second discrete nucleic acid targeting molecules that respectively target first and second portions of RNA transcribed from a target gene or genes, which can be the same or different, wherein the first nucleic acid targeting molecule modulates expression of the first portion of RNA, and the second nucleic acid targeting molecule modulates expression of the second portion of RNA.

The present invention also provides use of a construct as hereinbefore described, as a cosmetic.

The present invention also provides use of a construct as hereinbefore described, in research as a gene function analysis tool.

The present invention also provides a process of making a construct as hereinbefore described. Such a process typically comprises:
(i) synthesizing each of:
   (a) a first nucleic acid portion that is at least partially complementary to at least a first portion of RNA transcribed from a target gene;
   (b) a second nucleic acid portion that is at least partially complementary to at least a second portion of RNA transcribed from a target gene, which target gene may be the same or different to the target gene defined in (a);
   (c) a third nucleic acid portion that is at least partially complementary to the first nucleic acid portion of (a);
   (d) a fourth nucleic acid portion that is at least partially complementary to the second nucleic acid portion of (b);
(ii) contacting at least the first and second nucleic acid portions of (a) and (b) in vitro, so as to form a first nucleic acid duplex region comprising the first and second nucleic acid portions of (a) and (b);
(iii) contacting at least the third and fourth nucleic acid portions of (c) and (d) in vitro, so as to form a second nucleic acid duplex region comprising the third and fourth nucleic acid portions of (c) and (d);
(iv) forming a nucleic acid construct in vitro comprising at least the first and second nucleic acid duplex regions.

Preferably, a process according to the present invention further comprises generating from the construct at least first and second nucleic acid targeting molecules, wherein the first nucleic acid targeting molecule is capable of modulating expression of the target gene of (a), and comprises, or is derived from, at least the first nucleic acid portion of (a), and wherein the second nucleic acid targeting molecule is capable of modulating expression of the target gene of (b), and comprises, or is derived from, the second nucleic acid portion of (b). Typically, the at least first and second nucleic acid targeting molecules are generated subsequent to in vivo administration.

Preferably in a process according to the present invention labile functionality present in the construct is cleaved subsequent to in vivo administration so as to generate the at least first and second discrete nucleic acid targeting molecules. The labile functionality can comprise one or more unmodified nucleotides, whereby suitably the one or more unmodified nucleotides of the labile functionality represent one or more cleavage positions within the construct whereby subsequent to in vivo administration the construct is cleaved at the one or more cleavage positions so as to yield the at least first and second discrete nucleic acid targeting molecules.

Suitably in a process according to the present invention the cleavage positions are respectively located within the construct so that subsequent to cleavage the first discrete nucleic acid targeting molecule comprises, or is derived from, the first nucleic acid duplex region, and the second discrete nucleic acid targeting molecule comprises, or is derived from, the second nucleic acid duplex region.

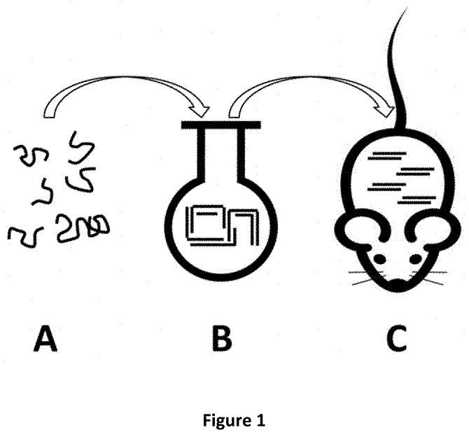

FIG. 1 is a schematic depiction of the fundamental concept of the multi-oligo nano-structures unit assembly (from individually synthesized separate oligonucleotide components) according to the present invention, its application and mode of action. (A) Initially, the individual oligonucleotides are synthesized separately following the design sequences and chemistries. (B) The oligonucleotides are then mixed in vitro (in the tube) in the conditions favoring formation of the nano-structures according to the predesigned scheme. (C) The formed nano-structures then are introduced into the cells or the whole organism, where, upon the exposure to biological environment (e.g. nucleases of the biological fluids and/or intra-cellularly), they disassemble to produce biologically active molecules, such as siRNAs or/and antisense oligonucleotides, capable to modulate expression (up- or down-regulate) of the target genes.

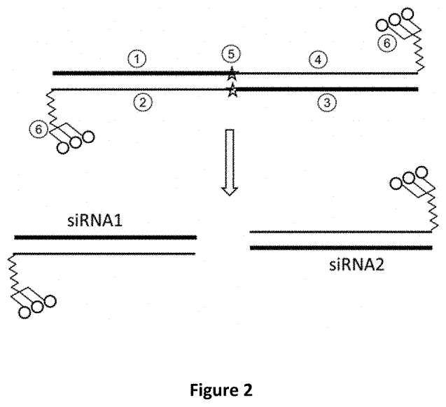

FIG. 2 provides an example according to the present invention of a relatively simple oligonucleotide nano-structure composed of 2-4 oligonucleotides. Segment (1) is complementary to the targeted sequence 1. Segment (2) is at least partially complementary to segment (1). Segment (3) is complementary to the targeted sequence 2, and segment (4) is at least partially complementary to the segment (3). Stars (5) represent the “liable” links between segments (1) and (4) and/or (2) and (3). In case segments (1), (2), (3) and (4) are chemically modified (e.g. with 2′F, 2′Ome, LNA modifications to increase resistance against nucleases), stars (5) could simply represent the unmodified RNA or DNA nucleotides. Otherwise, it could be some other linker. Component (6) represents the optional delivery moiety (e.g. GalNAc, Cholesterol, etc). The nano-structure depicted on the upper panel of the FIG. 2 is synthesized and assembled in vitro (in the tubes). Upon introduction into biological environment (exposure to extra- and/or intra-cellular biological fluids), the “liable” linkers/nucleotides are cleaved and the nano-structure disassembles into the functional gene expression modulating agents (e.g. siRNAs). In this particular case, two separate different siRNAs are generated (lower part of the FIG. 2).

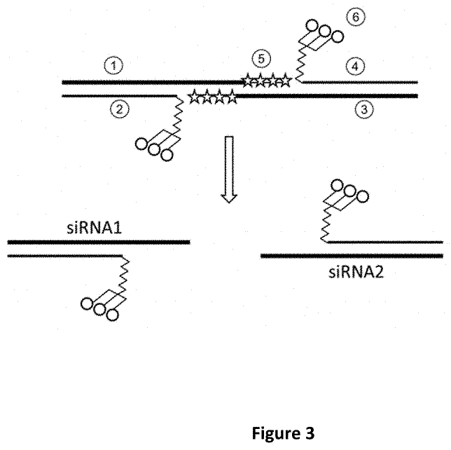

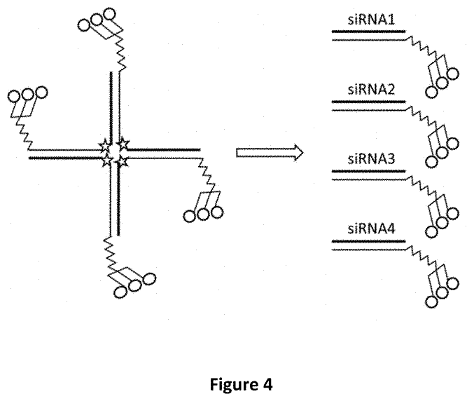

FIG. 3 provides another example of a multi-unit oligonucleotide nano-structure according to the present invention. It is somewhat similar to the structure depicted in FIG. 2, except that segments (1) and (4), as well as (2) and (3) are not physically (covalently) tied to each other. Thus the nano-structure is composed of four different oligo-nucleotide components. There is also partial complementarily between segments (1) and (3), in this case, also highlighted with stars (5). In case when segments (1), (2), (3) and (4) are chemically modified (e.g. by 2′F, 2″)Me, LNA modifications to increase stability against nucleases, stars (5) represent segments with “liable” positions (e.g. unmodified RNA or DNA nucleotides). In this particular case, the targeting/delivery moiety (6) (e.g. GalNAc, Cholesterol, etc) is attached (optionally) to different parts of the segments (2) and (4). The nano-structure depicted on the upper panel of the FIG. 3 is synthesized and assembled in vitro (in the tubes). Upon introduction into biological environment (exposure to extra- and/or intra-cellular biological fluids), the “liable” nucleotides are cleaved and the nano-structure disassembles into the functional gene expression modulating agents (e.g. siRNAs). In this particular case, two separate different siRNAs are generated (lower part of the FIG. 3). The passenger strands in such siRNAs would be somewhat shorter (could be as short as 8 nucleotides) than passenger strands in conventional siRNAs (18-21 nucleotides).

FIG. 4 provides another example of a multi-unit oligonucleotide nano-structure according to the present invention. It is conceptually similar to the nano-structure depicted in FIG. 2, except that it engages twice as many components. In this particular case, upon exposure to the biological environment, the nano-structure would disassemble into four different siRNAs.

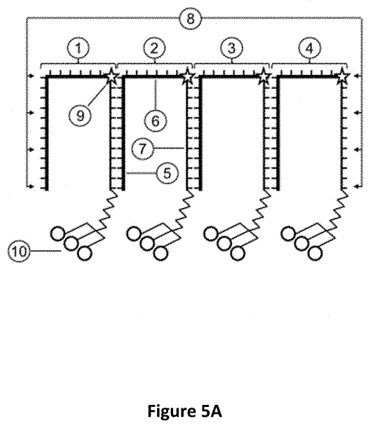

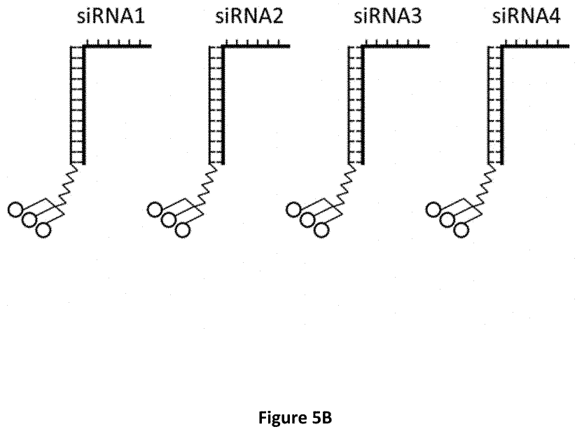

FIG. 5A is a schematic depiction of an example of a more complex and sophisticated multi-unit oligo-nucleotide nanostructure according to the present invention. As in previous examples, the nano-structure is aimed at being assembled in vitro (in the tube) from multiple oligonucleotide components, and to yield multiple active molecules (siRNAs in this case) upon exposure to the biological environment (e.g. introduced into animals, cells and exposed to extra- or/and intra-cellular biological fluids). The structure is composed of multiple individual oligonucleotides, with the total number of oligonucleotides varying from two and higher. For the convenience of visualizing the invention and in this particular scheme, the structure is composed of four oligonucleotides (1), (2), (3) and (4). Each of the oligonucleotides contains three segments: “targeting terminal segment” or TTS, as exemplified by (5), “targeting internal segment” or TIS, as exemplified by (6) and “adaptor terminal segment” or ATS, as exemplified by (7) for oligonucleotide (2). The neighboring oligonucleotides are connected to each other through complementary interactions between the TTS of one oligonucleotide and the ATS of another, neighboring oligonucleotide. The ATS of the last oligonucleotide (4), in this scheme, forms complimentary interactions with the TTS of the first oligonucleotide (1), which is schematically depicted by lines-and-arrows (8), to form a closed structure, in which each of the oligonucleotides is essentially equivalent to a building component. The TTS of each and every oligonucleotide starts with a 5′-terminus and the ATS of each and every oligonucleotide ends with a 3′-terminus. The length of each oligonucleotide may vary from 20 to 50 nucleotides, length of TTS—from 5 to 24 nucleotides, length of TIS—from 1 to 20 nucleotides, and length of ATS—from 5 to 24 nucleotides. The TTS and TIS of an individual and each oligonucleotide together comprise a contiguous sequence (highlighted with thicker line) at least partially complementary to a targeted sequence (e.g. mRNA, IncRNA, etc). In certain cases, sequence complementary to the targets can extend into the portion of or the entire ATS segment. The “liable” link depicted with the star (9) is incorporated in the junction of TIS and ATS of each of the building blocks. In case, when oligonucleotides are chemically modified to increase stability against the nucleases (e.g. using 2′F, 2′Ome, LNA, etc), the “liable” link could be simply non-modified nucleotide(s) (RNA or/and DNA). The construct may target different targets within the same targeted transcript (e.g. mRNA, IncRNA, etc), or different targeted sequences in different transcripts (e.g. mRNA, IncRNA, etc). In this particular case, the optional targeting/delivery moieties (10) (e.g. GalNAc, Cholesterol, etc) are attached to each of the building oligo-nucleotide blocks.

FIG. 5B depicts the outcome of the exposure of nano-constructs depicted on FIG. 5A to the biological environment (e.g. introduced into animals, cells and exposed to extra- or/and intra-cellular biological fluids). The “liable” links (stars (9) in FIG. 5A) would be attacked by nucleases, resulting in disassembly of the nano-structure into, in this particular case, four separate and different siRNAs. The final siRNAs might contain shorter passenger strands (as short as 8 nucleotides) than conventional siRNAs (18-21 nucleotides).

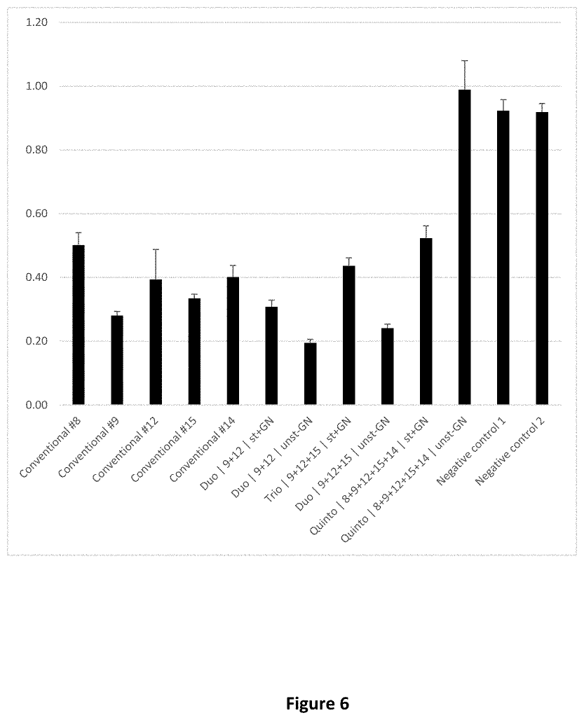

FIG. 6 shows the effect on mRNA expression of various constructs in Hep3B cells.

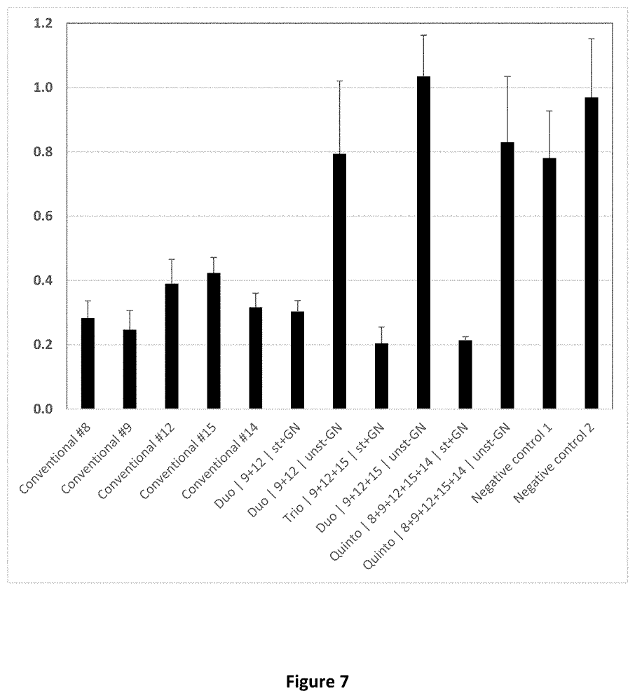

FIG. 7 shows the effect of single dose direct incubation of GalNAc-conjugated compounds in primary hepatocytes.

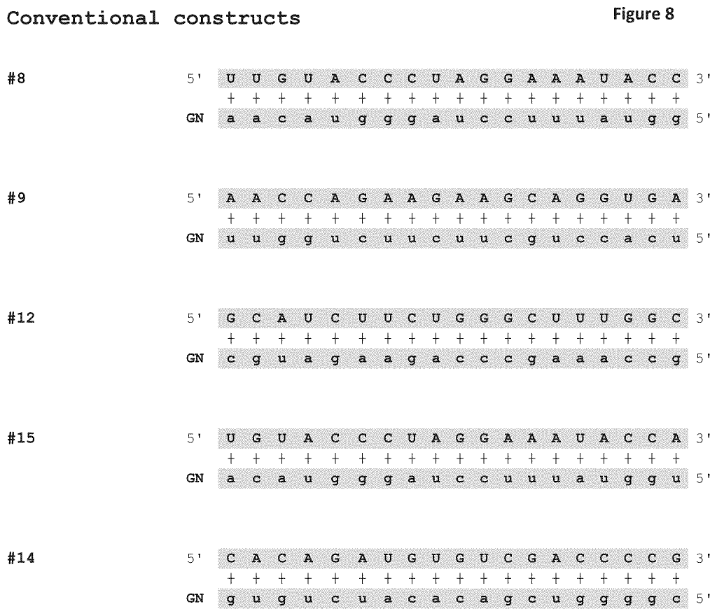

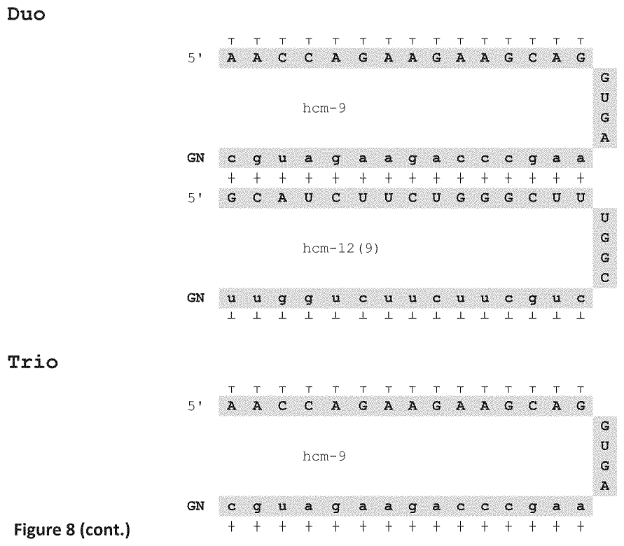

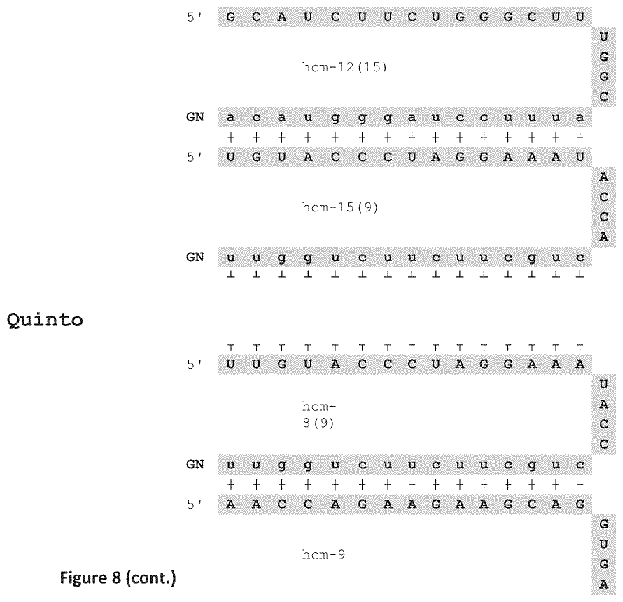

FIG. 8 shows the sequence of various conventional, duo, trio and quinto constructs.

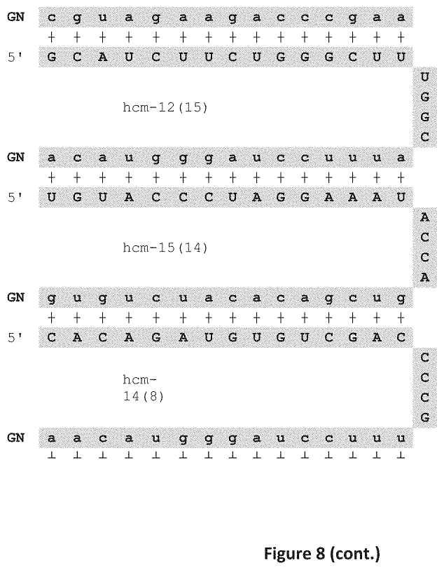

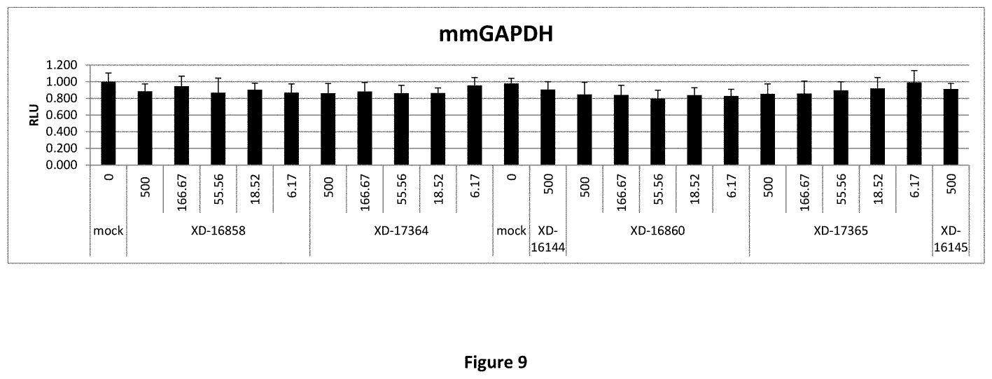

FIG. 9 shows the dose-response curves of various constructs against TMPRSS6.

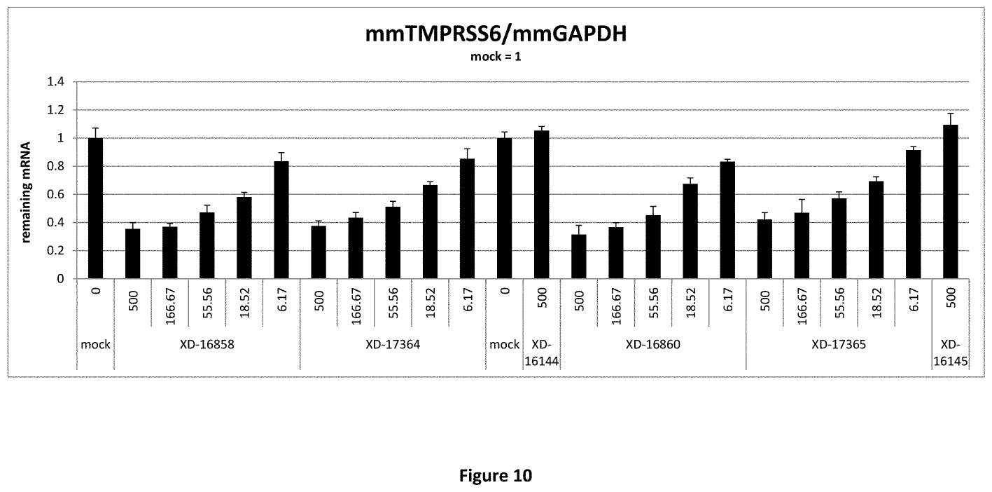

FIG. 10 shows the dose-response curves of various constructs against TMPRSS6.

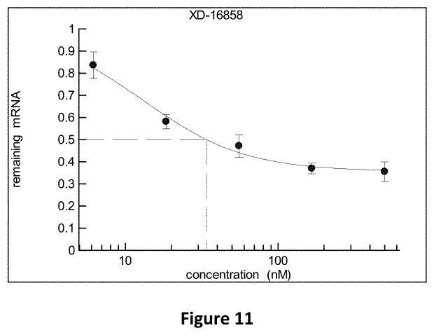

FIG. 11 shows the dose-response curve of construct XD-16858 against TMPRSS6.

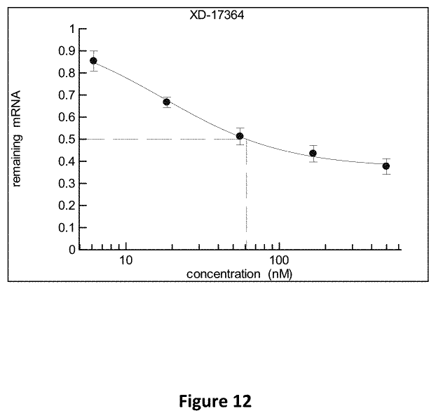

FIG. 12 shows the dose-response curve of construct XD-17364 against TMPRSS6.

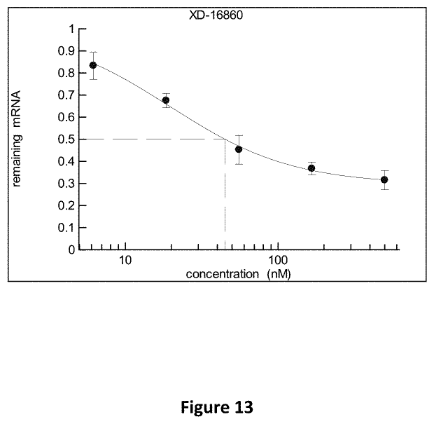

FIG. 13 shows the dose-response curve of construct XD-16880 against TMPRSS6.

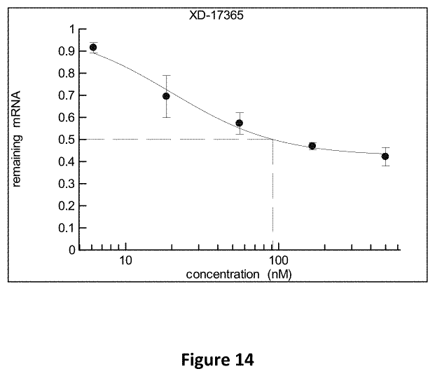

FIG. 14 shows the dose-response curve of construct XD-17365 against TMPRSS6.

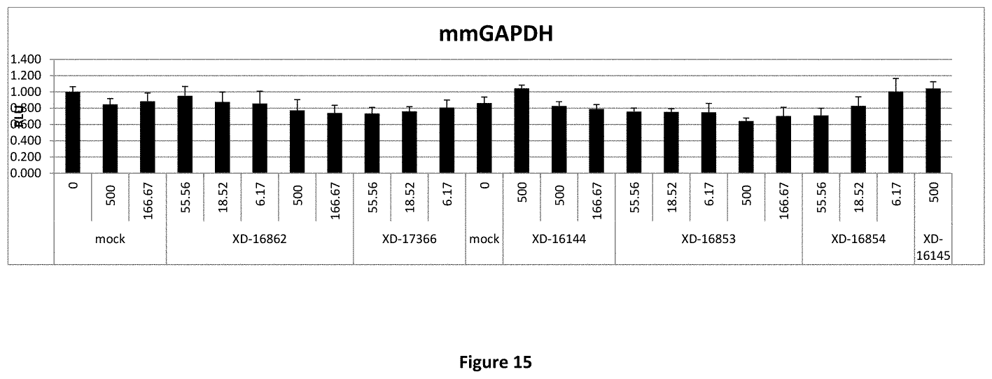

FIG. 15 shows the dose-response curves of various constructs against TMPRSS6.

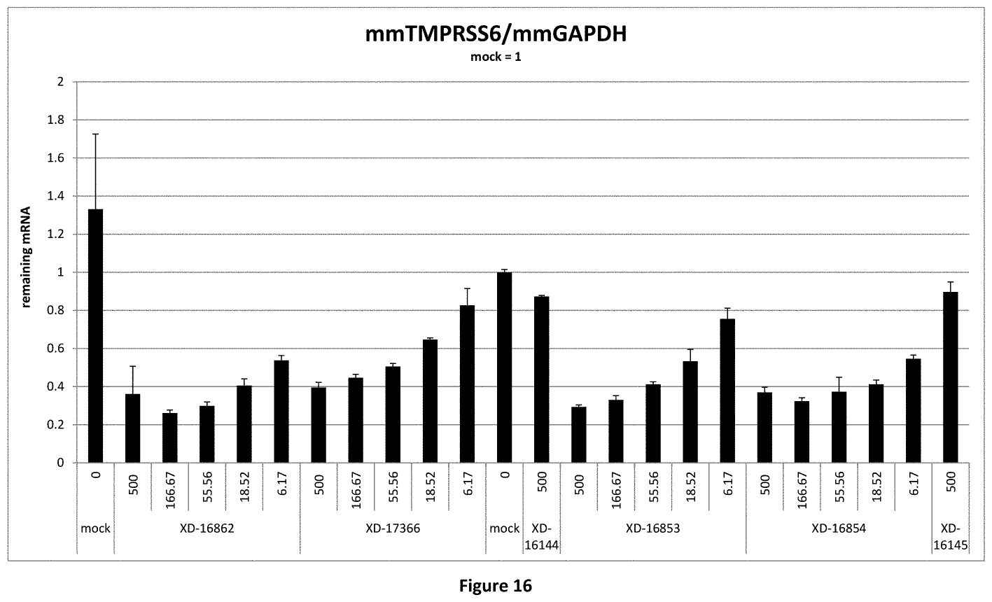

FIG. 16 shows the dose-response curves of various constructs against TMPRSS6.

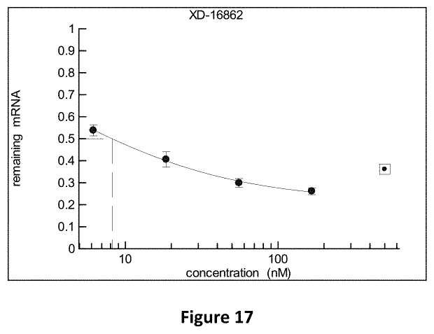

FIG. 17 shows the dose-response curve of construct XD-16862 against TMPRSS6.

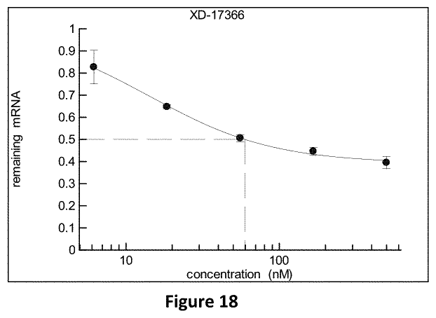

FIG. 18 shows the dose-response curve of construct XD-17366 against TMPRSS6.

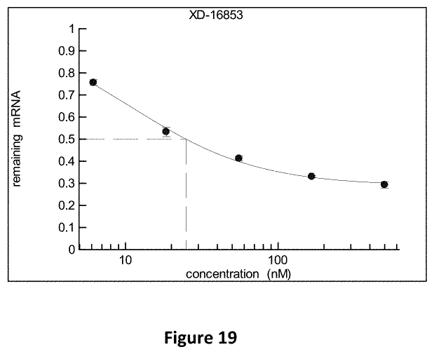

FIG. 19 shows the dose-response curve of construct XD-16853 against TMPRSS6.

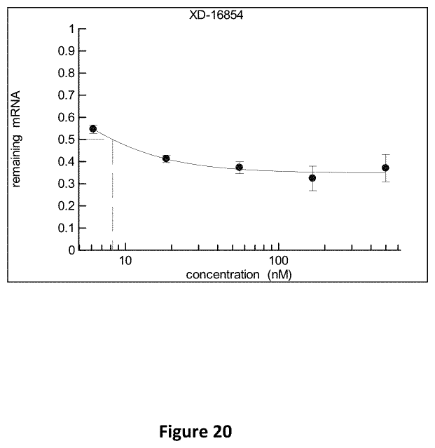

FIG. 20 shows the dose-response curve of construct XD-16854 against TMPRSS6.

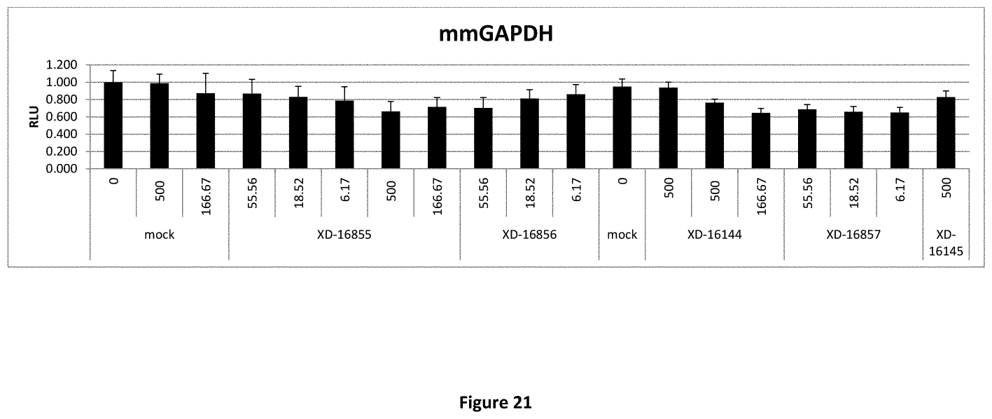

FIG. 21 shows the dose-response curves of various constructs against TMPRSS6.

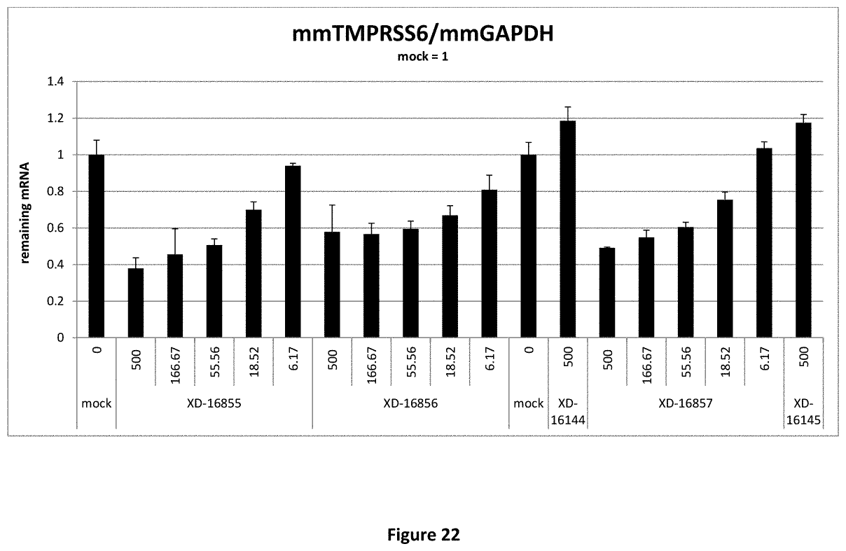

FIG. 22 shows the dose-response curves of various constructs against TMPRSS6.

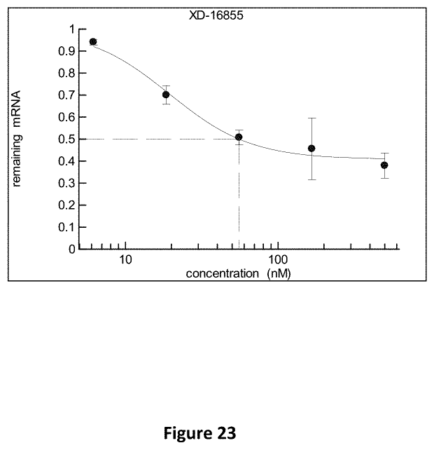

FIG. 23 shows the dose-response curve of construct XD-16855 against TMPRSS6.

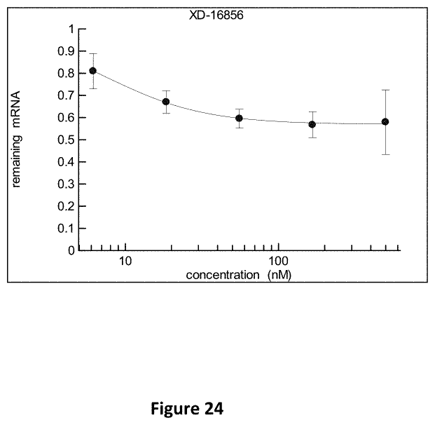

FIG. 24 shows the dose-response curve of construct XD-16856 against TMPRSS6.

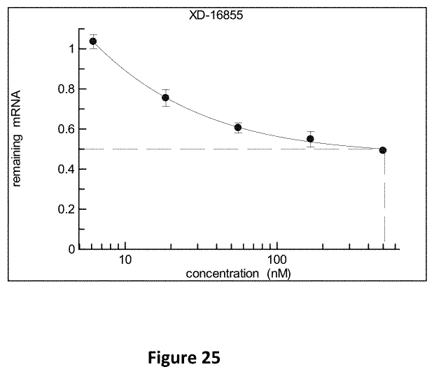

FIG. 25 shows the dose-response curve of construct XD-16855 against TMPRSS6.

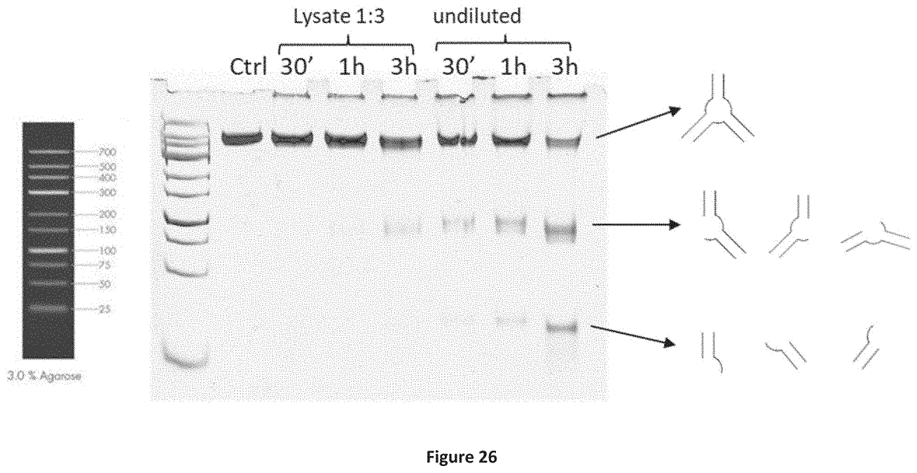

FIG. 26 shows time-dependent cleavage into single duplexes of a triple targeting conjugate (construct XD-16860) in liver lysosomal extract.

The terminology used herein is for the purpose of describing particular embodiments only and is not intended to be limiting of the invention. As used herein, the term “and/or” includes any and all combinations of one or more of the associated items. As used herein, the singular forms “a”, “an” and “the” are intended to include the plural forms as well as the singular forms, unless the context clearly indicates otherwise. It will be further understood that the terms “comprises” and/or “comprising” when used in this specification, specify the presence of stated features, steps, operations, elements and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art, to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of features, steps, operations, elements and/or components are disclosed. Each of these has individual benefit and each can also be used in conjunction with one or more, or in some cases all, of the other disclosed features, steps, operations, elements and/or components. Accordingly, for the sake of clarity, this description will refrain from repeating every possible combination of the individual features, steps, operations, elements and/or components in an unnecessary fashion. Nevertheless, the specifications should be read with the understanding that such combinations are entirely within the scope of the invention.

The above discussed specific Figures, and the following specific Examples and associated Tables and Figures, are for the purposes of explanation, with numerous specific details being set forth in order to provide a thorough understanding of the present invention. It will be evident, however, to one skilled in the art that the present invention may be practiced without these specific details and the claims as set forth herein are not therefore limited to such specific details. As such, this disclosure is to be considered as an exemplification of the invention, and is not intended to limit the invention to the specific embodiments illustrated by the Examples and Figures.

Exemplary features of constructs according to the present invention are as follows:

1) contain multiple (2 and more) oligonucleotides, tied together into nano-structure predominantly through complementary (Watson-Crick) interactions;

2) optionally, other (e.g.) covalent bindings may be recruited to build the nano-structures and/or add various ligands (e.g. delivery/targeting moieties);

3) the oligonucleotide constructs of the invention predominantly comprise chemically modified nucleotides (e.g. 2′F, 2′OMe, LNO, PNA, MOE, BNA, PMO, phosphorothioate, phosphodithioate, etc.), mostly (but not only) to increase resistance to nucleases;

4) nano-structures are likely (but not necessarily) to contain “liable” components (e.g. chemical linkers, unmodified nucleotides, etc), which would allow the nano-structures to disassemble upon exposure to certain biologic environments (e.g. exposure to extra- and/or intra-cellular fluids); particular examples could be (but not limited): a) cleavage of the oligo backbone by nucleases in the sites with non-modified nucleotides; b) cleavage of the chemical linkage due to the change of pH (e.g. in endosomes);

5) nano-structures are expected to disassemble upon exposure to certain biologic environment to release the active components (e.g. siRNA, antisense oligonucleotides, small molecules, peptides, etc) to modulate (up- or down-regulate) target gene expression in cells/organisms;

6) nano-structures are likely (but not necessarily) to contain the delivery/targeting moieties (e.g. GalNAc and or other carbohydrates, cholesterol, peptides, small molecules, others), attached through the linkers (or by other means) to the particles;

7) nano-structures can be used to modulation gene expression to study gene function, to treat various diseases, or for other applications, including, but not limited to cosmetics and/or agriculture.

The present invention therefore includes nano-structures comprising multiple oligonucleotides self-assembled through complementary interactions comprising oligonucleotides having sequences complementary to one or multiple genes. In one particular embodiment of the invention the nano-structures are capable of disassembling into simpler structures (e.g. individual oligonucleotides or duplexes) in biological environment (e.g. inside the organism and/or inside the cell). The present invention also includes compositions comprising such nano-structures and methods of using the same for modulation of gene expression to study gene function, to treat various diseases, or for other applications, including, but not limited to cosmetics and/or agriculture.

Aspects of the invention are demonstrated by the following non-limiting examples.

### EXAMPLES

Tables 3 and 4, and FIG. 8, set out sequences, and constructs formed therefrom, as used in the following Examples.

### Example 1: Single Dose Transfection in Hep3B Cells

Hep3B cells were incubated in 96-well plates at a density of 15,000 cells per each well. The compounds tested with this study were at a final concentration of 50 nM. Reverse transfection was carried out using Lipofectamine 2000 at 0.5 μL per well. In addition to the test compounds two controls ((XD-10064) TTR-directed siRNA and (XD-00033) aha-1 directed siRNA) were also used. The duration of incubation was 24 hours. Subsequently mRNA was isolated and quantified using a bDNA assay (Quantigene 1.0/2.0).

A summary of the results obtained from this experiment are presented in Table 1 and FIG. 6.

TABLE 1
Summary of results for Example 1

TMPRSS6

| Compound ID | mean | SD |
|---|---|---|
| Conventional #8 | 0.50 | 0.04 |
| Conventional #9 | 0.28 | 0.01 |
| Conventional #12 | 0.39 | 0.09 |
| Conventional #15 | 0.33 | 0.01 |
| Conventional #14 | 0.40 | 0.04 |
| Duo \| 9 + 12 \| st + GN | 0.31 | 0.02 |
| Duo \| 9 + 12 \| unst − GN | 0.20 | 0.01 |
| Trio \| 9 + 12 + 15 \| st + GN | 0.44 | 0.02 |
| Duo \| 9 + 12 + 15 \| unst − GN | 0.24 | 0.01 |
| Quinto \| 8 + 9 + 12 + 15 + 14 \| st + GN | 0.52 | 0.04 |
| Quinto \| 8 + 9 + 12 + 15 + 14 \| unst − GN | 0.99 | 0.09 |
| Negative control 1 | 0.92 | 0.03 |
| Negative control 2 | 0.92 | 0.03 |

“unst”—un-stabilized against nucleases
“st”—stabilized against nucleases
“−GN”—without the GalNAc moiety
“+GN”—with the GalNAc moiety

### Example 2: Simile Dose Direct Incubation of GalNAc-Conjugated Compounds in Primary Hepatocytes

Primary mouse hepatocytes (Lot #MC830; ThermoFisher Scientific) were incubated in a 96-well plate at a density of 45,000 cells per well. The compounds tested with this study were added at a final concentration of 500 nM. In addition to the test compounds two controls (XD-12171) TTR-directed siRNA and (XD-00033) aha-1 directed siRNA (no Galnac used as a negative control) were also used. A direct incubation transfection (without transfection lipid) method was used. The duration of incubation was 72 hours. Subsequently mRNA was isolated and quantified using a bDNA assay (Quantigene 1.0/2.0).

A summary of the results obtained from this experiment are presented in Table 2 and FIG. 7.

TABLE 2
Summary of results for Example 2

TMPRSS6

| Compound ID | mean | SD |
|---|---|---|
| Conventional #8 | 0.28 | 0.05 |
| Conventional #9 | 0.25 | 0.06 |
| Conventional #12 | 0.39 | 0.08 |
| Conventional #15 | 0.42 | 0.05 |
| Conventional #14 | 0.32 | 0.04 |
| Duo \| 9 + 12 \| st + GN | 0.30 | 0.03 |
| Duo \| 9 + 12 \| unst − GN | 0.79 | 0.23 |
| Trio \| 9 + 12 + 15 \| st + GN | 0.20 | 0.05 |
| Duo \| 9 + 12 + 15 \| unst − GN | 1.03 | 0.13 |
| Quinto \| 8 + 9 + 12 + 15 + 14 \| st + GN | 0.21 | 0.01 |
| Quinto \| 8 + 9 + 12 + 15 + 14 \| unst − GN | 0.83 | 0.20 |
| Negative control 1 | 0.78 | 0.15 |
| Negative control 2 | 0.97 | 0.18 |

“unst”—un-stabilized against nucleases
“st”—stabilized against nucleases
“−GN”—without the GalNAc moiety
“+GN”—with the GalNAc moiety

### Example 3: Dose Response Curves

Dose-response curves of constructs according to the present invention directed against TMPRSS6, with and without a cleavage site, are shown in FIGS. 9 to 25. IC50/KD results are summarized in Table 5.

The results were obtained further to direct incubation of GalNAc-conjugated compounds in primary mouse hepatocytes, 60,000 cells/well. Concentrations employed were 500, 166.67, 55.56, 18.52 and 6.17 nM, by direct incubation for 72 hours.

### Example 4: Trio Treated with Liver Lysosome Extract Disassembles into Individual Components

A triple targeting conjugate according to the present invention based on Seq ID No 11 plus Seq ID No 15 plus Seq ID No 16, construct XD-16860 as set out in Table 4, was incubated in liver lysosomal extract (Xenotech) to show cleavage into single duplexes that is expected to happen after uptake of the constructs in the hepatocyte cells.

Incubation conditions were as follows:

A) lysate 1:3 diluted, incubation time 30 min, 1 h, 3 h

B) Undiluted lysate, incubation time 30 min, 1 h, 3 h

Electrophoresis conditions were as follows:

Non-denaturing 20% acrylamide gels, 1×TBE-buffer, GelRed stain.

The results are shown in FIG. 26.

TABLE 3
Single-stranded oligonucleotides used

| SEQ ID NO. (s) | Sequence |
|---|---|
| 1 | puUfgUfaCfcCfuAfgGfaAfaUfaCfc |
| 2 | GfgUfaUfuUfCfCfuAfgGfgUfaCfaAf(NHC6)(GalNAc3) |
| 3 | paAfcCfaGfaAfgAfaGfcAfgGfuGfa |
| 4 | UfcAfcCfuGfCfUfuCfuUfcUfgGfuUf(NHC6)(GalNAc3) |
| 5 | pgCfaUfcUfuCfuGfgGfcUfuUfgGfc |
| 6 | GfcCfaAfaGfCfCfcAfgAfaGfaUfgCf(NHC6)(GalNAc3) |
| 7 | puGfuAfcCfcUfaGfgAfaAfuAfcCfa |
| 8 | UfgGfuAfuUfUfCfcUfaGfgGfuAfcAf(NHC6)(GalNAc3) |
| 9 | pcAfcAfgAfuGfuGfuCfgAfcCfcCfg |
| 10 | CfgGfgGfuCfGfAfcAfcAfuCfuGfuGf(NHC6)(GalNAc3) |
| 11 | paAfcCfaGfaAfgAfaGfcAfgsGfsusGfsAAfaGfCfCfcAfgAfaGfaUfgCf(NHC6)(GalNAc3) |
| 12 | pgCfaUfcUfuCfuGfgGfcUfusUfsgsGfsCCfuGfCfUfuCfuUfcUfgGfuUf(NHC6)(GalNAc3) |
| 13 | pAACCAGAAGAAGCAGGUGAAAGCCCAGAAGAUGC |
| 14 | pGCAUCUUCUGGGCUUUGGCCUGCUUCUUCUGGUU |
| 15 | pgCfaUfcUfuCfuGfgGfcUfusUfsgsGfsCAfuUfUfCfcUfaGfgGfuAfcAf(NHC6)(GalNAc3) |
| 16 | puGfuAfcCfcUfaGfgAfaAfusAfscsCfsACfuGfCfUfuCfuUfcUfgGfuUf(NHC6)(GalNAc3) |
| 17 | pGCAUCUUCUGGGCUUUGGCAUUUCCUAGGGUACA |
| 18 | pUGUACCCUAGGAAAUACCACUGCUUCUUCUGGUU |
| 19 | puGfuAfcCfcUfaGfgAfaAfusAfscsCfsAGfuCfGfAfcAfcAfuCfuGfuGf(NHC6)(GalNAc3) |
| 20 | pcAfcAfgAfuGfuGfuCfgAfcsCfscsCfsGUfuUfCfCfuAfgGfgUfaCfaAf(NHC6)(GalNAc3) |
| 21 | PuUfgUfaCfcCfuAfgGfaAfasUfsasCfsCCfuGfCfUfuCfuUfcUfgGfuUf(NHC6)(GalNAc3) |
| 22 | pUGUACCCUAGGAAAUACCAGUCGACACAUCUGUG |
| 23 | pCACAGAUGUGUCGACCCCGUUUCCUAGGGUACAA |
| 24 | pUUGUACCCUAGGAAAUACCCUGCUUCUUCUGGUU |
| 25 | paAfcCfaGfaAfgAfaGfcAfgsGfsusGfsaAfaGfCfCfcAfgAfaGfaUfgCf(NHC6)(GalNAc3) |
| 26 | pgCfaUfcUfuCfuGfgGfcUfusUfsgsGfscCfuGfCfUfuCfuUfcUfgGfuUf(NHC6)(GalNAc3) |
| 27 | pgCfaUfcUfuCfuGfgGfcUfusUfsgsGfscAfuUfUfCfcUfaGfgGfuAfcAf(NHC6)(GalNAc3) |
| 28 | puGfuAfcCfcUfaGfgAfaAfusAfscsCfsaCfuGfCfUfuCfuUfcUfgGfuUf(NHC6)(GalNAc3) |
| 29 | puGfuAfcCfcUfaGfgAfaAfusAfscsCfsaGfuCfGfAfcAfcAfuCfuGfuGf(NHC6)(GalNAc3) |
| 30 | pcAfcAfgAfuGfuGfuCfgAfcsCfscsCfsgUfuUfCfCfuAfgGfgUfaCfaAf(NHC6)(GalNAc3) |
| 31 | puUfgUfaCfcCfuAfgGfaAfasUfsasCfscCfuGfCfUfuCfuUfcUfgGfuUf(NHC6)(GalNAc3) |

Table 3 key
p = phosphate
u, a, c, g = 2′-methyl modified
Uf, Af, Cf, Cf = 2′-fluoro modified
U, A, C, G = unmodified
s = phosphorothioate
(NHC6) = linker
(GalNAc3) = trivalent N-acetylgalactosamine

TABLE 4
Construct key based on single-stranded
oligonucleotides used in this study

| | | |
|---|---|---|
| XD-16853 | Conventional #8 | Seq ID No 1 plus |
| | | Seq ID No 2 |
| XD-16854 | Conventional #9 | Seq ID No 3 plus |
| | | Seq ID No 4 |
| XD-16855 | Conventional #12 | Seq ID No 5 plus |
| | | Seq ID No 6 |
| XD-16856 | Conventional #15 | Seq ID No 7 plus |
| | | Seq ID No 8 |
| XD-16857 | Conventional #14 | Seq ID No 9 plus |
| | | Seq ID No 10 |
| XD-16858 | Duo/9 + 12/st + GN | Seq ID No 11 plus |
| | | Seq ID No 12 |
| | Duo/9 + 12/unst − GN | Seq ID No 13 plus |
| | | Seq ID No 14 |
| XD-16860 | Trio/9 + 12 + 15/st + GN | Seq ID No 11 plus |
| | | Seq ID No 15 plus |
| | | Seq ID No 16 |
| | Trio/9 + 12 + 15/unst − GN | Seq ID No 13 plus |
| | | Seq ID No 17 plus |
| | | Seq ID No 18 |
| XD-16862 | Quinto/8 + 9 + 12 + 15 + 14/st + GN | Seq ID No 21, plus |
| | | Seq ID No 11 plus |
| | | Seq ID No 15 plus |
| | | Seq ID No 19 plus |
| | | Seq ID No 20 |
| | Quinto/8 + 9 + 12 + 15 + 14/unst − GN | Seq ID No 13 plus |
| | | Seq ID No 17 plus |
| | | plus Seq ID No 27 |
| | | Seq ID No 22 plus |
| | | Seq ID No 23 plus |
| | | Seq ID No 24 |
| XD-17364 | | Seq ID No 25 plus |
| | | Seq ID No 26 |
| XD-17365 | | Seq ID No 25 plus |
| | | Seq ID No 27 plus |
| | | Seq ID No 28 |
| XD-17366 | | Seq ID No 25 plus |
| | | Seq ID No 27 plus |
| | | Seq ID No 29 plus |
| | | Seq ID No 30 plus |
| | | Seq ID No 31 |

TABLE 5
IC50/KD results

| siRNA ID | IC₅₀ (nM) | max. KD (%) |
|---|---|---|
| XD-16853 | 24.92 | 71% |
| XD-16854 | 8.25 | 68% |
| XD-16855 | 55.43 | 62% |
| XD-16856 | n.a. | 42% |
| XD-16857 | 511.81 | 51% |
| XD-16858 | 34.03 | 64% |
| XD-16860 | 44.97 | 68% |
| XD-16862 | 8.19 | 74% |
| XD-17364 | 61.19 | 62% |
| XD-17365 | 91.67 | 58% |
| XD-17366 | 59.58 | 60% |

1. A nucleic acid construct comprising at least:
(a) a first nucleic acid portion that is at least partially complementary to at least a first portion of RNA transcribed from a target gene;
(b) a second nucleic acid portion that is at least partially complementary to at least a second portion of RNA transcribed from a target gene, which target gene may be the same or different to the target gene defined in (a);
(c) a third nucleic acid portion that is at least partially complementary to said first nucleic acid portion of (a), so as to form a first nucleic acid duplex region therewith;
(d) a fourth nucleic acid portion that is at least partially complementary to said second nucleic acid portion of (b), so as to form a second nucleic acid duplex region therewith;
wherein said construct is designed such that subsequent to in vivo administration said construct disassembles to yield at least first and second discrete nucleic acid targeting molecules that respectively target said RNA portions transcribed from said target genes of (a) and (b);
whereby (i) said first nucleic acid targeting molecule is capable of modulating expression of said target gene of (a), and comprises, or is derived from, at least said first nucleic acid portion of (a), and (ii) said second nucleic acid targeting molecule is capable of modulating expression of said target gene of (b), and comprises, or is derived from, said second nucleic acid portion of (b).

2. A construct according to claim 1, wherein said construct is designed to disassemble such that said first and second discrete nucleic acid targeting molecules are respectively processed by independent RNAi-induced silencing complexes.

3. A construct according to claim 1, which further comprises labile functionality such that subsequent to in vivo administration said construct is cleaved so as to yield said at least first and second discrete nucleic acid targeting molecules.

4. A construct according to claim 3, wherein said labile functionality comprises one or more unmodified nucleotides.

5. A construct according to claim 4, wherein said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules.

6. A construct according to claim 5, wherein said cleavage positions are respectively located within the construct so that subsequent to cleavage said first discrete nucleic acid targeting molecule comprises, or is derived from, said first nucleic acid duplex region, and said second discrete nucleic acid targeting molecule comprises, or is derived from, said second nucleic acid duplex region.

7. A construct according to claim 1, wherein said first nucleic acid portion of (a) is directly or indirectly linked to said fourth nucleic acid portion of (d) as a primary structure.

8. A construct according claim 1, which is a dual targeting construct and wherein said second nucleic acid portion of (b) is directly or indirectly linked to said third nucleic acid portion of (c) as a primary structure.

9. A construct according claim 1, that further comprises 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and wherein each of the 1 to 8 additional nucleic acid portions respectively form additional duplex regions with respective passenger nucleic acid portions that are respectively at least partially complementary therewith.

10. A construct according to claim 9, wherein said second nucleic acid portion of (b), and said 1 to 8 additional nucleic acid portions, are directly or indirectly linked to selected passenger nucleic acid portions as respective primary structures.

11. A construct according to claim 1, wherein said direct or indirect linking represents either (i) an internucleotide nick, (ii) an internucleotide bond, or (iii) a nucleic acid linker portion of 1 to 10 nucleotides, wherein in the case of (i) there exists some complementarity between the first nucleic acid portion of (a) and the second nucleic acid portion of (b), or the third nucleic acid portion of (c) and the fourth nucleic acid portion of (d).

12. A construct according to claim 1, represented by the following schematic structure: 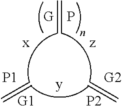
wherein
G1 represents said first nucleic acid portion of (a);
G2 represents said second nucleic acid portion of (b);
P1 represents said third nucleic acid portion of (c);
P2 represents said fourth nucleic acid portion of (d);
G represents said 1 to 8 additional nucleic acid portions that are respectively at least partially complementary to an additional 1 to 8 portions of RNA transcribed from one or more target genes;
P represents said passenger nucleic acid portions that are respectively at least partially complementary with said 1 to 8 additional nucleic acid portions and forming said duplex regions therewith;
each of G1, G2, P1, P2 can each respectively include the same or different numbers of nucleotides;
n is an integer selected between 0 to 8;
wherein there is present one or more adjacent and/or non-adjacent cleavage positions, that at least allows disassembly of at least G1 from P2, and/or at least G2 from P1, and when n is 1 to 8 there is also present one or more adjacent and/or non-adjacent cleavage positions that allows disassembly of at least G2 from an adjacent P, and/or at least P1 from an adjacent G;
each of x, y, z either represent (i) an internucleotide nick, (ii) a internucleotide bond, or (iii) a nucleic acid linker portion of 1 to 10 nucleotides;
wherein when n is 0, and x, y, z represent an internucleotide nick between G1 and P2, and P1 and G2 respectively, then there exists some complementarity between either G1 and G2, or P1 and P2.

13. A construct according to claim 11, wherein said nucleic acid linker portion is single stranded.

14. A construct according to claim 1, which further comprises one or more ligands, typically conjugated to said third nucleic acid portion of (c), and/or said fourth nucleic acid portion of (d).

15. A construct according to claim 14, wherein said first nucleic acid portion of (a), and/or said second nucleic acid portion of (b), and/or said third nucleic acid portion of (c), and/or said fourth nucleic acid portion of (d), respectively have a 5′ to 3′ directionality thereby defining 5′ and 3′ regions thereof, and wherein said one or more ligands are conjugated at the 3′ region of any of (i) said third nucleic acid portion of (c), and/or (ii) said fourth nucleic acid portion of (d).

16. A construct according to claim 14, wherein said third nucleic acid portion of (c), and/or said fourth nucleic acid portion of (d), respectively have a 5′ to 3′ directionality thereby defining 5′ and 3′ regions thereof, and wherein said one or more ligands are conjugated at one or more regions intermediate of the 5′ and 3′ regions thereof.

17. A construct according to claim 1, wherein said one or more ligands are any cell directing moiety, such as lipids, carbohydrates, aptamers, vitamins and/or peptides that bind cellular membrane or a specific target on cellular surface.

18. A construct according to claim 17, wherein said one or more ligands comprise one or more carbohydrates.

19. A construct according to claim 18, wherein said one or more carbohydrates can be a monosaccharide, disaccharide, trisaccharide, tetrasaccharide, oligosaccharide or polysaccharide.

20. A construct according to claim 19, wherein said one or more carbohydrates comprise one or more galactose moieties, one or more lactose moieties, one or more N-Acetyl-Galactosamine moieties, and/or one or more mannose moieties.

21. A construct according to claim 20, wherein said one or more carbohydrates comprise one or more N-Acetyl-Galactosamine moieties.

22. A construct according to claim 21, which comprises two or three N-Acetyl-Galactosamine moieties.

23. A construct according to claim 14, wherein said one or more ligands are attached in a linear configuration, or in a branched configuration.

24. A construct according to claim 23, wherein said one or more ligands are attached as a biantennary or triantennary configuration, or as a configuration based on single ligands at different positions.

25. A construct according to claim 1, wherein said first nucleic acid portion of (a), and/or said second nucleic acid portion of (b), and/or said third nucleic acid portion of (c), and/or said fourth nucleic acid portion of (d), are respectively 7 to 20 nucleotides in length, preferably 10 to 18 nucleotides in length, more preferably about 15 nucleotides in length.

26. A construct according to claim 1, wherein said nucleic acid linker portion is 1 to 8 nucleotides in length, preferably 2 to 6 nucleotides in length, more preferably about 4 nucleotides in length.

27. A construct according to claim 1, which further comprises one or more phosphorothioate or phosphorodithioate internucleotide linkages.

28. A construct according to claim 27, which comprises 1 to 15 phosphorothioate or phosphorodithioate internucleotide linkages.

29. A construct according to claim 27, which comprises one or more phosphorothioate or phosphorodithioate internucleotide linkages at one or more of the 5′ and/or 3′ regions of said first nucleic acid portion of (a), and/or said second nucleic acid portion of (b), and/or said third nucleic acid portion of (c), and/or said fourth nucleic acid portion of (d).

30. A construct according to claim 27, which comprises phosphorothioate or phosphorodithioate internucleotide linkages between at least two adjacent nucleotides of the nucleic acid linker portion.

31. A construct according to claim 30, which comprises a phosphorothioate or phosphorodithioate internucleotide linkage between each adjacent nucleotide that is present in said nucleic acid linker portion.

32. (canceled)

33. A construct according to claim 1, wherein at least one nucleotide of at least one of the following is modified:
the first nucleic acid portion of (a); and/or
the second nucleic acid portion of (b); and/or
the third nucleic acid portion of (c); and/or
the fourth nucleic acid portion of (d).

34. A construct according to claim 33, wherein one or more of the odd numbered nucleotides starting from the 5′ region of one of the following are modified, and/or wherein one or more of the even numbered nucleotides starting from the 5′ region of one of the following are modified, wherein typically the modification of the even numbered nucleotides is a second modification that is different from the modification of odd numbered nucleotides:
the first nucleic acid portion of (a); and/or
the second nucleic acid portion of (b); and/or
the third nucleic acid portion of (c); and/or
the fourth nucleic acid portion of (d).

35. A construct according to claim 33, wherein one or more of the odd numbered nucleotides starting from the 3′ region of the third nucleic acid portion of (c) are modified by a modification that is different from the modification of odd numbered nucleotides starting from the 5′ region of the first nucleic acid portion of (a); and/or
wherein one or more of the odd numbered nucleotides starting from the 3′ region of the fourth nucleic acid portion of (d) are modified by a modification that is different from the modification of odd numbered nucleotides starting from the 5′ region of the second nucleic acid portion of (b).

36. A construct according to claim 33, wherein one or more of the even numbered nucleotides starting from the 3′ region of: (i) the third nucleic acid portion of (c), and/or (ii) the fourth nucleic acid portion of (d) are modified by a modification that is different from the modification of odd numbered nucleotides starting from the 3′ region of these respective portions.

37. A construct according to claim 33, wherein at least one or more of the modified even numbered nucleotides of (i) the first nucleic acid portion of (a), and/or (ii) the second nucleic acid portion of (b) is adjacent to at least one or more differently modified odd numbered nucleotides of these respective portions.

38. A construct according to claim 33, wherein at least one or more of the modified even numbered nucleotides of (i) the third nucleic acid portion of (c), and/or (ii) the fourth nucleic acid portion of (d), is adjacent to at least one or more differently modified odd numbered nucleotides of these respective portions.

39. A construct according to claim 33, wherein a plurality of adjacent nucleotides of (i) the first nucleic acid portion of (a), and/or (ii) the second nucleic acid portion of (b), are modified by a common modification.

40. A construct according to claim 33, wherein a plurality of adjacent nucleotides of (i) the third nucleic acid portion of (c), and/or (ii) the fourth nucleic acid portion of (d), are modified by a common modification.

41. A construct according to claim 39, wherein said plurality of adjacent commonly modified nucleotides are 2 to 4 adjacent nucleotides, or 3 or 4 adjacent nucleotides.

42. A construct according to claim 41, wherein said plurality of adjacent commonly modified nucleotides are located in the 5′ region of (i) the third nucleic acid portion of (c), and/or (ii) the fourth nucleic acid portion of (d).

43. A construct according to claim 11 wherein a plurality of adjacent commonly modified nucleotides are located in the nucleic acid linker portion.

44. A construct according to claim 33, wherein the one or more of the modified nucleotides of first nucleic acid portion of (a) do not have a common modification present in the corresponding nucleotide of the third nucleic acid portion of (c) of the first duplex region; and/or one or more of the modified nucleotides of second nucleic acid portion of (b) do not have a common modification present in the corresponding nucleotide of the fourth nucleic acid portion of (d) of the second duplex region; do not have a common modification present in the corresponding nucleotide of the corresponding passenger nucleic acid portions of the respective duplex regions.

45. A construct according to claim 33, wherein the one or more of the modified nucleotides of the first nucleic acid portion of (a) are shifted by at least one nucleotide relative to a commonly modified nucleotide of the third nucleic acid portion of (c); and/or one or more of the modified nucleotides of the second nucleic acid portion of (b) are shifted by at least one nucleotide relative to a commonly modified nucleotide of the fourth nucleic acid portion of (d) are shifted by at least one nucleotide relative to a commonly modified nucleotide of the passenger nucleic acid portions as defined in claim 9, 10 or 12.

46. A construct according to claim 33, wherein the modification and/or modifications are each and individually sugar, backbone or base modifications, and are suitably selected from the group consisting of 3′-terminal deoxy-thymine, 2′-O-methyl, a 2′-deoxy-modification, a 2′-amino-modification, a 2′-alkyl-modification, a morpholino modification, a phosphoramidate modification, phosphorothioate or phosphorodithioate group modification, a 5′ phosphate or 5′ phosphate mimic modification and a cholesteryl derivative or a dodecanoic acid bisdecylamide group modification.

47. A construct according to claim 33, wherein the modification is any one of a locked nucleotide, an abasic nucleotide or a non-natural base comprising nucleotide.

48. A construct according to claim 33, wherein at least one modification is a 2′-O-methyl modification in a ribose moiety.

49. A construct according to claim 33, wherein at least one modification is a 2′-F modification in a ribose moiety.

50. A construct according to claim 33 wherein the nucleotides at any of positions 2 and 14 downstream from the first nucleotide of the 5′ region of (i) the first nucleic acid portion of (a); and/or (ii) the second nucleic acid portion of (b) do not contain 2′-O-methyl modifications in ribose moieties.

51. A construct according to claim 33, wherein the nucleotides of (i) the third nucleic acid portion of (c); and or (ii) the fourth nucleic acid portion of (d) that respectively correspond in position to any of the nucleotides at any of positions 11 to 13 downstream from the first nucleotide of the 5′ region of (i) the first nucleic acid portion of (a); and/or (ii) the second nucleic acid portion of (b); do not contain 2′-O-methyl modifications in ribose moieties.

52. A construct according to claim 50, wherein the nucleotides at any of positions 2 and 14 downstream from the first of (i) the first nucleic acid portion of (a); and/or (ii) the second nucleic acid portion of (b); contain 2′-F modifications in ribose moieties.

53. A construct according to claim 50, wherein the nucleotides of (i) the third nucleic acid portion of (c); and or (ii) the fourth nucleic acid portion of (d); that respectively correspond in position to any of the nucleotides at any of positions 11 to 13 downstream from the first nucleotide of the 5′ region of (i) the first nucleic acid portion of (a); and/or (ii) the second nucleic acid portion of (b) contain 2′-F modifications in ribose moieties.

54. A construct according to claim 1, which comprises one or more unmodified nucleotides.

55. A construct according to claim 54, wherein said one or more unmodified nucleotides can replace any modified nucleotide.

56. (canceled)

57. A conjugate according to claim 51, wherein
all nucleotides other than
the unmodified nucleotides; and/or
the nucleotides at any of positions 2 and 14 downstream from the first nucleotide of the 5′ region of (i) the first nucleic acid portion of (a); and/or (ii) the second nucleic acid portion of (b) and/or
the nucleotides of (i) the third nucleic acid portion of (c); and or (ii) the fourth nucleic acid portion of (d);
contain 2′-O-methyl modifications in ribose moieties.

58. A construct according to claim 1, which comprises at least one vinylphosphonate modification, such as at least one vinylphosphonate modification in the 5′ region of (i) the first nucleic acid portion of (a); and/or (ii) the second nucleic acid portion of (b).

59. A construct according to claim 1, wherein
one or more nucleotides of
the first nucleic acid portion of (a); and/or
the second nucleic acid portion of (b); and/or
the third nucleic acid portion of (c); and/or
the fourth nucleic acid portion of (d)
is an inverted nucleotide and is attached to the adjacent nucleotide via the 3′ carbon of the nucleotide and the 3′ carbon of the adjacent nucleotide, and/or is an inverted nucleotide and is attached to the adjacent nucleotide via the 5′ carbon of the nucleotide and the 5′ carbon of the adjacent nucleotide.

60. A construct according to claim 59, wherein the inverted nucleotide is attached to the adjacent nucleotide via a phosphate group by way of a phosphodiester linkage; or is attached to the adjacent nucleotide via a phosphorothioate group; or is attached to the adjacent nucleotide via a phosphorodithioate group.

61. A construct according to claim 1, which is blunt ended.

62. A conjugate according to claim 1, wherein
the first nucleic acid portion of (a); and/or
the second nucleic acid portion of (b); and/or
the third nucleic acid portion of (c); and/or
the fourth nucleic acid portion of (d)
has an overhang.

63. A construct according to claim 1, wherein the target RNA is selected from at least one of: mRNA, lncRNA, and/or other RNA molecules.

64. A composition comprising a construct according to claim 1, and a physiologically acceptable excipient.

65. A construct according to claim 1, for use in the treatment of a disease or disorder.

66. Use of a construct according to claim 1, in the manufacture of a medicament for treating a disease or disorder.

67. A method of treating a disease or disorder comprising administration of a construct according to claim 1, to an individual in need of treatment.

68. A method according to claim 67, wherein the construct is administered subcutaneously or intravenously to the individual.

69. A method according to claim 67, wherein subsequent to in vivo administration the construct disassembles to yield at least first and second discrete nucleic acid targeting molecules that respectively target first and second portions of RNA transcribed from a target gene or genes, which can be the same or different, wherein the first nucleic acid targeting molecule modulates expression of the first portion of RNA, and the second nucleic acid targeting molecule modulates expression of the second portion of RNA.

70-72. (canceled)

73. A process of making a construct according to claim 1, which comprises:
(i) synthesizing each of:
(a) a first nucleic acid portion that is at least partially complementary to at least a first portion of RNA transcribed from a target gene;
(b) a second nucleic acid portion that is at least partially complementary to at least a second portion of RNA transcribed from a target gene, which target gene may be the same or different to the target gene defined in (a);
(c) a third nucleic acid portion that is at least partially complementary to said first nucleic acid portion of (a);
(d) a fourth nucleic acid portion that is at least partially complementary to said second nucleic acid portion of (b);
(ii) contacting at least said first and second nucleic acid portions of (a) and (b) in vitro, so as to form a first nucleic acid duplex region comprising said first and second nucleic acid portions of (a) and (b);
(iii) contacting at least said third and fourth nucleic acid portions of (c) and (d) in vitro, so as to form a second nucleic acid duplex region comprising said third and fourth nucleic acid portions of (c) and (d);
(iv) forming a nucleic acid construct in vitro comprising at least said first and second nucleic acid duplex regions.

74. A process according to claim 73, which further comprises generating from said construct at least first and second nucleic acid targeting molecules, wherein the first nucleic acid targeting molecule is capable of modulating expression of the target gene of (a), and comprises, or is derived from, at least the first nucleic acid portion of (a), and wherein the second nucleic acid targeting molecule is capable of modulating expression of said target gene of (b), and comprises, or is derived from, the second nucleic acid portion of (b).

75. A process according to claim 74, wherein said at least first and second nucleic acid targeting molecules are generated subsequent to in vivo administration.

76. A process according to claim 75, wherein labile functionality present in said construct is cleaved subsequent to in vivo administration so as to generate said at least first and second discrete nucleic acid targeting molecules.

77. A process according to claim 76, wherein said labile functionality comprises one or more unmodified nucleotides.

78. A process according to claim 77, wherein said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules.

79. A process according to claim 78, wherein said cleavage positions are respectively located within the construct so that subsequent to cleavage said first discrete nucleic acid targeting molecule comprises, or is derived from, said first nucleic acid duplex region, and said second discrete nucleic acid targeting molecule comprises, or is derived from, said second nucleic acid duplex region.
